(19) United States
(12) Patent Application Publication
(10) Pub. No.: US 2019/0284290 A1
(43) Pub. Date: Sep. 19, 2019

(54) DR5 RECEPTOR AGONIST COMBINATIONS

(71) Applicant: Amgen Inc., Thousand Oaks, CA (US)

(72) Inventors: Pamela Mary Holland, Belmont, MA (US); Jonathan David Graves, Belmont, MA (US); Jennifer Joy Kordich, Ada, MI (US); Julia Catherine Piasecki, Seattle, WA (US); Ian Nevin Foltz, Burnaby (CA)

(21) Appl. No.: 16/176,343

(22) Filed: Oct. 31, 2018

Related U.S. Application Data
(63) Continuation of application No. 14/388,762, filed on Sep. 26, 2014, now abandoned.
(60) Provisional application No. 61/616,929, filed on Mar. 28, 2012.

(51) Int. Cl. C07K 16/28; A61K 39/395; A61K 38/19

(52) U.S. Cl. CPC C07K 16/2878; A61K 39/39541; A61K 2039/505; A61K 38/191; A61K 39/3955; A61K 39/39558

(57) ABSTRACT

The present invention includes apoptotic compositions and methods for inducing apoptosis of cancer cells independent of NK cells. An apoptotic composition comprises a cooperative combination of antibodies that specifically bind to human DR5, or a cooperative combination of an anti-DR5 antibody and TRAIL. Administration of therapeutically effective amounts of an apoptotic composition induces apoptosis of apoptosis sensitive cancer cells.

### CROSS-REFERENCE TO RELATED APPLICATIONS

This application is a continuation application of U.S. patent application Ser. No. 14/388,762, filed Sep. 26, 2014, which is a national stage application under 35 U.S.C. § 371 of International Application No. PCT/US2013/034163, having an international filing date of Mar. 27, 2013, which claims the benefit of U.S. Provisional Application No. 61/616,929, filed Mar. 28, 2012, all of which are incorporated herein by reference.

### REFERENCE TO THE SEQUENCE LISTING

The present application is being filed along with a Sequence Listing in electronic format. The Sequence Listing is provided as a file entitled A-1685-US-CNT_Sequence_Listing_ST25.txt created Oct. 29, 2018, which is 12 KB in size. The information in the electronic format of the Sequence Listing is incorporated herein by reference in its entirety.

### FIELD OF THE INVENTION

The present invention relates to compositions and methods of inhibiting the growth of cancer cells in a mammal by using a combination of therapeutic agents that specifically bind to DR5 receptors expressed by cancer cells and act cooperatively to induce cell death in these cancer cells.

### BACKGROUND OF THE INVENTION

TRAIL (TNF-Related Apoptosis Inducing Ligand), is a member of the TNF (Tumor Necrosis Factor) family of cytokines. In common with several other members of the TNF family, TRAIL is homotrimeric, exists as both surface-bound and soluble forms, and binds to several receptors that are members of the TNF-receptor superfamily. Two of these receptors, DR4 (Death Receptor 4) and DR5 (Death Receptor 5) are capable of generating an apoptotic signal. Binding of TRAIL to DR4 or DR5 at the surface of a sensitive target cell induces receptor clustering, a step that is critical for the formation of a productive a death-inducing signaling complex, and subsequent apoptosis of the target cell.

Certain agonistic antibodies directed against DR4 or DR5 can also induce death of target cells. However, highly purified preparations of these antibodies, that are essentially free of aggregates, require additional cross-linking in order to induce optimal receptor clustering and apoptosis. This cross-linking can be provided in vitro by multi-valent agents that bind the Fc (factor crystallizable) region of antibodies, such as anti-IgG antibodies or protein G. See, e.g., Miller et al, J. Immunol., 170: 4854-4861 (2003). In preclinical animal models, the activity of agonistic anti-DR4 or DR5 antibodies is highly dependent on their interaction with receptors that bind the Fc region of IgG, suggesting that cross-linking in vivo is mediated by these Fcγ receptors. Work by ourselves and others has shown that the critical FcγRs for mediating anti-DR5 antibody crosslinking in humans are likely to be FcγRII and FcγRIIIA that are expressed primarily on myeloid and natural killer (NK) cells populations respectively. See, e.g., Wilson et al., Cancer Cell 19:101-113 (2011).

The therapeutic potential of apoptosis induced by the TRAIL receptor system has been investigated in human clinical trials with soluble recombinant human TRAIL or agonist antibodies directed against either DR4 or DR5. Although Phase 1 and 1 b combination studies provided encouraging preliminary results, findings from randomized Phase 2 studies have failed to demonstrate an overall statistically significant clinical benefit from death receptor agonist therapies. While the reason(s) for these results are not completely understood, one possible explanation is that neither soluble TRAIL, nor anti-DR5 antibodies, induced optimal receptor clustering and therefore apoptotic signaling in patients.

In the case of anti-DR antibodies, the requirement for interaction with FcγR-bearing cells may have limited their efficacy. For example, FcγR-bearing cells in the microenvironment of a particular tumor may be low and/or reduced as a result of chemotherapeutic toxicity. Additionally, polymorphic variation in the FCGR3A receptor on NK cells at the locus F158V (SNP ID: rs396991) can lead to significant differences in binding affinity between an antibody's Fc domain and NK cells and, as a corollary, result in significant differences in the ability of such antibodies to induce apoptosis via FcγRIIIA binding. This variability is particularly problematic as most human patients are not homozygous for the high-affinity FCGR3A V158 allele.

Reports of synergy between an anti-DR5 antibody (AD5-10) and TRAIL in inducing apoptosis of tumor cells in vitro has been reported. Guo et al. (Journal of Biological Chemistry, 280(51): 41940-41952 (2005)). However, subsequent investigation by EI-Gazzar et al. utilizing AD5-10 found that the function of natural killer (NK) cells is necessary for the activation of DR5-mediated apoptosis. EI-Gazzar et al., Mol. Cancer Ther., 9(4): 1007-1018 (2010). In that study, in an NK-competent human ovarian cancer xenograft mouse model the tumor was eradicated but in NK-depleted mice cytotoxic activity of AD5-10 was abolished.

Therefore, what is needed in the art are agonistic anti-DR5 therapeutics that can induce effective receptor clustering in the absence of a requirement for exogenous cross-linking. Here agonistic combinations of TRAIL and anti-DR binding polypeptides or of pairs of binding polypeptides are provided that synergistically interact to induce apoptosis via DR4 and/or DR5. Thus, the present invention provides potent agonists that are independent of the requirement for interaction with immune cells.

### SUMMARY OF THE INVENTION

The present invention includes a composition for inducing DR5-mediated apoptosis of a mammalian cancer cell expressing this receptor. This apoptotic composition includes a first anti-DR5 binding polypeptide that specifically binds to the extracellular domain of a DR5 receptor of the cancer cell. The composition also includes at least one of a second anti-DR5 binding polypeptide that specifically binds to the extracellular domain of the DR5 receptor. The first binding polypeptide and the second binding polypeptide do not competitively inhibit each other from specifically binding to their cognate receptor(s). Specific binding of the first binding polypeptide and the second binding polypeptide act to cooperatively induce apoptosis of the mammalian cancer cell without requiring natural killer (NK) cell mediated cross-linking. Alternatively, or in addition, the apoptotic composition can comprise the first binding polypeptide and a tumor necrosis factor related apoptosis inducing ligand (TRAIL) or a TRAIL variant that specifically binds to the DR5 receptor. TRAIL or its variant and the first binding polypeptide do not competitively inhibit each other from specifically binding their cognate receptor. Specific binding of these molecules cooperatively induces apoptosis of the mammalian cancer cell without requiring natural killer (NK) mediation of the apoptosis. In some embodiments the mammalian cancer cell is a human cancer cell. In some embodiments, the first anti-DR5 binding polypeptide and/or the second anti-DR5 binding polypeptide is an antibody, which can be a fully human antibody. In some embodiments, at least one of the first or second anti-DR5 binding polypeptides do not specifically bind to FCGR3A of NK cells. The apoptotic compositions of the invention can be administered in a therapeutically effective amount to inhibit the growth of DR5 expressing cancer in a mammal such as a human. The composition can be administered in combination with a therapeutically effective amount of a chemotherapeutic agent.

### BRIEF DESCRIPTION OF THE DRAWINGS

FIG. 1 shows that an anti-DR5 antibody (AMG 655) and a version that is incapable of binding Fc receptors (N297A) can both cooperate with TRAIL to induce apoptosis of tumor cells in the H460 tumor xenograft model. Neither the wild-type or mutant AMG 655 show any single agent activity in this model.

FIG. 2A shows that an anti-DR5 antibody (AMG 655) potentiates TRAIL and LZ-TRAIL mediated killing of WM35 cells in the absence of exogenous cross-linking.

FIG. 2B shows histograms of surface DR4 and DR5 expression levels on WM35 cells analyzed by flow cytometry.

FIG. 3 shows that an anti-DR5 antibody (AMG 655) cooperates with TRAIL to induce apoptosis in multiple tumor cell lines.

FIG. 4 shows that an anti-DR5 antibody (AMG 655) cooperates with TRAIL to induce apoptosis in lung tumor cell lines that are sensitive to the antibody but resistant to TRAIL.

FIG. 5 shows that in the H838 lung cancer cell line an anti-DR5 antibody (AMG 655) and TRAIL cooperate to induce increased cell killing at all doses over either agent alone.

FIGS. 6A and 6C show that an anti-DR5 antibody (AMG 655) and TRAIL do not cooperate to induce apoptosis in normal human monocytes or epithelial cells, respectively.

FIGS. 6B and 6D show histograms of DR5 expression levels on the relevant cell type.

FIG. 7 shows that only a subset of anti-DR5 antibodies are capable of cooperating with TRAIL to induce apoptosis.

FIG. 8 shows a 2-dimensional matrix of anti-DR5 antibodies, the bin of selected antibodies, and that certain pairs of antibodies exhibit cooperativity.

FIG. 9 shows the relative in vitro killing activity of anti-DR5 antibody pairs or anti-DR5 antibodies plus TRAIL in Colo205 and H460 cells.

FIG. 10 shows cooperativity between two IgG2 anti-DR5 antibodies that do not interact with Fc receptors in the Colo205 tumor xenograft model.

### DETAILED DESCRIPTION

The present invention relates, in part, to compositions and methods for inducing apoptosis of DR5 and/or DR4 expressing mammalian cancer cells. The combinations of the present invention can induce apoptosis without requiring specific binding to FCGR3A and thus without requiring NK cell mediated induction of apoptosis. The present invention provides combinations that can act to mediate apoptosis via both NK cell-dependent and NK cell-independent pathways. Thus, the present invention provides for a more effective anti-cancer therapeutic than NK cell-dependent compositions which are otherwise susceptible to variations in the amount of NK cells in the tumor microenvironment as well as to differences in affinity of IgG1 antibodies to polymorphic forms of FCGR3A.

The section headings are used herein for organizational purposes only, and are not to be construed as in any way limiting the subject matter described. The disclosure of all patents, patent applications, and other documents cited herein are hereby expressly incorporated by reference in their entirety. Unless specific definitions are provided, the nomenclature utilized in connection with, and the laboratory procedures and techniques of biochemistry and molecular biology described herein are those well known and commonly used in the art. Standard techniques may be used for biochemical syntheses, biochemical analyses, pharmaceutical preparation, formulation, and delivery, and treatment of patients. The definitions provided extend to the defined terms and variations thereof.

The term “afucosylation” or “afucosylated” in the context of an Fc, such as an Fc-polypeptide, refers to a substantial lack of a fucose moiety covalently attached, directly or indirectly, to residue 297 of the human IgG1 Fc numbered according to the EU index (Kabat et al., Sequences of Proteins of Immunological Interest, 5th Ed. Public Health Service, National Institutes of Health, Bethesda, Md. (1991)), or the corresponding residue in non-IgG1 or non-human IgG1 immunoglobulins. Thus, in a composition comprising a plurality of afucosylated Fc-polypeptides at least 70% of the Fc-polypeptides will be not be fucosylated, directly or indirectly (e.g., via intervening sugars) at residue 297 of the Fc, and in some embodiments at least 80%, 85%, 90%, 95%, or 99% will not be fucosylated, directly or indirectly at residue 297 of the Fc. Those of skill will recognize that in the context of the present invention it may be possible to create a biologically inactive fucosylation of an Fc that is biologically similar to afucosylation in increasing affinity to FCGR3A. Such constructs are to be understood as included within the scope of the defined term.

The term “apoptosis” refers generally to a specific programmed cell death inducing mechanism initiated by an agonist of a DR4 and/or DR5 receptor. While not bound by theory, cell death induced by agonizm of these receptors is thought to proceed via apoptosis. However, the defined term is not limited to that specific mechanism and may proceed via other forms of programmed cell death.

The term “agonist” or “agonistic” or “agonize” in the context of a binding polypeptide's activity, refers to its function in mediating induction of apoptosis via the DR5 and/or DR4 receptor in an apoptosis sensitive mammalian cancer cell, such as a human cancer cell, which expresses DR4 and/or DR5 on the cell surface. An exemplary human cancer cell sensitive to apoptosis is Colo205 (ATCC CCL-222). A DR5 agonist will induce apoptosis via DR5, a DR4 agonist will induce apoptosis via DR4, a dual DR5/DR4 agonist (e.g., TRAIL ligand or a bispecific agonistic anti-DR4/DR5 antibody) is able to induce apoptosis through both DR4 and DR5. Whether apoptotic induction is mediated via DR5 and/or DR4 can be determined using methods and reagents known in the art. Thus, for example, apoptosis sensitive DR specific cell lines are known in the art. An exemplary DR5(+)/DR4(−) cell line is WM35 (ATCC CRL-2807). An exemplary DR5(−)/DR4(+) cell line is ST486 (ATCC CRL 1647).

The term “antibody” includes reference to isolated forms of both glycosylated and non-glycosylated immunoglobulins of any isotype or subclass, including any combination of: 1) human, humanized, and chimeric antibodies, 2) monospecific (e.g., DR5 or DR4) or multi-specific antibodies (e.g., DR4 and DR5), and 3) monoclonal or polyclonal antibodies, irrespective of whether such antibodies are produced, in whole or in part, via immunization, through recombinant technology, by way of in vitro synthetic means, or otherwise. Thus, the term “antibody” is inclusive of antibodies that are prepared, expressed, created or isolated by recombinant means, such as (a) antibodies isolated from an animal (e.g., a mouse) that is transgenic for human immunoglobulin genes, or a B-cell or hybridoma prepared therefrom, (b) antibodies isolated from a host cell transfected to express the antibody (e.g., from a transfectoma), (c) antibodies isolated from a recombinant combinatorial antibody library, and (d) antibodies prepared, expressed, created or isolated by any other means that involve splicing of immunoglobulin gene sequences to other DNA sequences. Antibodies are also inclusive of antibody fragments such as Fab, F(ab′)₂, scFv (single-chain Fv), and derivatives such as diabodies. In some embodiments the antibodies of the present invention are monoclonal antibodies, such as humanized or human monoclonal antibodies. Typically, antibodies of the present invention will be IgG1 or IgG2 subclass antibodies. The antibody may bind its target with a Kd of less than about 10 nM, 5 nM, 1 nM, or 500 pM.

The term “binding polypeptide” means a polypeptide that specifically binds to a target, such as mammalian DR5 and/or DR4. A binding polypeptide that specifically binds to DR5 is referred to as an “anti-DR5 binding polypeptide” while a binding polypeptide that specifically binds to DR4 is referred to as an “anti-DR4 binding polypeptide.” A binding polypeptide that specifically binds to both DR5 and DR4 (such as a bispecific molecule) is referred to as an “anti-DR4/anti-DR5 binding polypeptide.” A binding polypeptide can be derivitized to increase, for example, solubility or pharmacokinetic properties. Often the binding polypeptide includes an Fc (fragment crystallizable) that can bind to FCGR3A, such as a human IgG1 Fc. Exemplary binding polypeptides include antibodies, peptibodies, and Fc-polypeptides.

The term “competitively inhibit” means a measurable inhibition (partial or complete) of specific binding of one species of binding polypeptides to a particular target due to specific binding of a second species of binding polypeptide to the target. A variety of methods are known to quantify the extent of competition such as competition ELISA (enzyme linked immunosorbent assay) assays. See, Buxton et al., Infect Immun. 27(2):405-10 (1980).

The term “cooperate”, “cooperative”, or “cooperatively” in the context of of induction of cell death (e.g., apoptosis) by a combination of therapeutic agents (i.e., an “apoptotic composition”), such as binding polypeptides, means that the activity of the combination in inducing cell death is greater than the activity of any one agent. Thus, cooperativity can be at least partially-additive, at least fully-additive, or greater than fully-additive (i.e., synergistic) of the activity of each agent in the combination. Cooperativity can be assessed in terms of efficacy, potency, or both.

The term “cross-linking” means the clustering of DR5 and/or DR4 receptors on a cell which expresses one or both receptors. Without being bound by theory, it is believed that clustering of the death receptors plays a role in induction of apoptosis. Cross-linking can be achieved in vitro using exogenous cross-linking agents. Cross-linking can be achieved in vivo using endogenous (i.e., naturally occurring) cross-linkers such as FCGR3A NK cells or macrophages.

The terms “derivation” or “derivatives” refer to modification of a binding polypeptide (such as an antibody) and/or chemotherapeutic agent by covalently linking it, directly or indirectly, so as to alter such characteristics as half-life, bioavailability, immunogenicity, solubility, toxicity, potency, or efficacy while retaining or enhancing its therapeutic benefit. Derivatives can be made by glycosylation, pegylation, and lipidation, or by protein conjugation. Exemplary derivitizing agents include a linear polymer (e.g., polyethylene glycol (PEG), polylysine, dextran, etc.); a branched-chain polymer (See, for example, U.S. Pat. No. 4,289,872 to Denkenwalter et al., issued Sep. 15, 1981; U.S. Pat. No. 5,229,490 to Tam, issued Jul. 20, 1993; WO 93/21259 by Frechet et al., published 28 Oct. 1993); a lipid or liposome; a cholesterol group (such as a steroid); a carbohydrate or oligosaccharide.

The terms “DR4” or “death receptor 4” or “TRAIL-R1” or “TR-1” refer to the 468 amino acid polypeptide set forth in SEQ ID NO: 2 of U.S. Pat. No. 6,342,363 (incorporated herein by reference) as well as related native (i.e., wild-type) human polypeptides such as allelic variants, splice variants, and mature forms of the polypeptide (i.e., lacking a leader sequence). The terms “DR4” or “death receptor 4” or “TRAIL-R1” or “TR-1” in reference to a non-human mammal(s) refers to the homologous receptor of that mammal.

The term “DR5” or TRAIL-R″ or “Apo-2” or “TR-2” or “TRAIL Receptor-2” refer to the 440 amino acid polypeptide set forth in SEQ ID NO: 2 of U.S. Pat. No. 7,528,239 as well as related native (i.e., wild-type) human polypeptides such as allelic variants or splice variants such as, but not limited to, the 411 amino acid isoform set forth in SEQ ID NO: 1 in U.S. Pat. No. 6,342,369, and at SEQ ID NO: 2 of U.S. Pat. No. 6,743,625 (each patent incorporated herein by reference), including mature forms of the polypeptide (i.e., lacking a leader sequence). The term “DR5” or TRAIL-R″ or “Apo-2” or “TR-2” or “TRAIL Receptor-2” in reference to a non-human mammal(s) refers to the homologous receptor of that mammal.

The terms “effective amount” or “therapeutically effective amount” refer to a quantity and/or concentration of a binding polypeptide that when administered ex vivo (by contact with a cancer cell from a patient) or in vivo (by administration into a patient) for treatment of a DR5 (and/or DR4) sensitive cancer either alone (i.e., as a monotherapy) or in combination with a chemotherapeutic agent yields a statistically significant inhibition of cancer progression. In some embodiments the patient is a human patient. As used herein, the terms “treatment” or, “inhibit,” “inhibiting” or “inhibition” of cancer refers to at least one of: a statistically significant decrease in the rate of tumor growth, a cessation of tumor growth, or a reduction in the size, mass, metabolic activity, or volume of the tumor, as measured by standard criteria such as, but not limited to, the Response Evaluation Criteria for Solid Tumors (RECIST), or a statistically significant increase in progression free survival (PFS) or overall survival (OS).

The term “Fc” in the context of an “Fc-polypeptide” refers to the Fc (fragment crystallizable) of an immunoglobulin that specifically binds to human FCGR3A or its homolog in a non-human mammal. An Fc can be a naturally-occurring (“native”) human IgG1 Fc but also includes truncated forms of IgG1 Fc (“truncated Fc”) that specifically bind to FCGR3A (or its homologs), or variants of naturally-occurring IgG1 Fc (“Fc variants”) made by substitution, deletion, or addition of amino acid residues wherein the variant Fc specifically binds to FCGR3A. A truncated Fc can be at least 80%, 85%, 90%, 95%, 96%, 97%, 98%, or 99% of the full-length Fc. The number of substitutions, deletions, or additions of a truncated Fc or of an Fc variant can be up to 1, 2, 3, 4, 5, 6, 7, 8, 9, 10, 15, or 20. Specific binding of a truncated Fc or Fc variant to FCGR3A is generally at least 80%, 85%, 90% or 95% of native Fc specific binding as quantified by such methods as ELISA.

The terms “FCGR3A” or “CD16a” “Fcγ receptor IIIA” or “FcγRIIIA” means the human Fc receptor of the same designation. A bi-allelic polymorphism of the human IgG receptor FcγRIIIA (CD16a) termed “F158V” can be distinguished by virtue of the presence of the amino acid valine (V) or phenylalanine (F) at the locus identified at the National Center for Biotechnology Information (NCBI) Single Nucleotide Polymorphism (SNP) database at cluster report rs396991. These two alleleic forms are commonly referred to in the literature and herein as “valine158” or “V158” for the polymorphism having the residue valine at the rs396991 SNP locus of human FcγRIIIA, and “phenylalanine158” or “F158” for the polymorphism having the residue phenylalanine at the rs396991 SNP locus of human FcγRIIIA. See also, Leppers-van de Straat et al., J. Immunological Methods, 242: 127-132 (2000) and Ravetch and Perussia, J. Exp. Med., 170:481-497 (1989).

The term “Fc-polypeptide” refers to the product of a covalent attachment between an Fc and at least one binding polypeptide that specifically binds to DR5 (an anti-DR5 binding polypeptide) and/or DR4 (an anti-DR4 binding polypeptide). The fusion of Fc and polypeptide may be via a direct covalent bond (e.g., via a peptide bond) or indirect covalent bond (e.g., via artificial chemical linker). In some embodiments the Fc-polypeptide is an agonistic Fc-polypeptide. Exemplary Fc-polypeptides include antibodies, peptibodies (WO 2000/24782, incorporated herein by reference), avimers (Nature Biotechnology 23, 1556-1561 (2005), Fc-soluble receptor conjugates, Covx-bodies ((WO 2008/056346; antibodies conjugated to targeting peptides, e.g., U.S. Pat. No. 7,521,425, incorporated herein by reference), or a cytotoxin, or a therapeutic (e.g., an antibody drug conjugate (“ADC”)), or an Fc-human TRAIL ligand fusion. In some embodiments, the Fc-polypeptide is bivalent. In some embodiments, the Fc-polypeptide is bivalent and bispecific. In some embodiments, the Fc-polypeptide is a homodimer comprising two IgG1 Fc and in some embodiments the Fc-polypeptide is a heterodimer comprising one IgG1 Fc and one non-IgG1 Fc. In some embodiments the homodimer and heterodimers are fully human antibodies.

The term “high-affinity” in the context of an Fc-polypeptide, means that the Fc is modified or constructed such that it specifically binds to human FCGR3A expressed by a native cell (e.g., a human NK cell) that is homozygous for the F158 allele with at least the same affinity as at least one of: an identical but afucosylated human Fc-polypeptide (e.g., an antibody), or an identical human Fc-polypeptide comprising a modification to increase FCGR3A affinity at residue 332 (per EU index of Kabat; see, U.S. Pat. No. 7,317,091 and/or U.S. Pat. No. 7,662,925) such as a isoleucine to glutamic acid substitution. Generally, a high-affinity Fc-polypeptide specifically binds to human FCGR3A with at least the same affinity as a native fucosylated Fc-polypeptide specifically binds to human FCGR3A expressed by a native cell homozygous for the V158 allele. Means to measure binding affinity are known in the art and include but are not limited to competition assays such as an AlphaLISA™ (Perkin Elmer, Mass. USA) ELISA assay. See, Poulsen, J., et al. 2007. J. Biomol Screen. 12:240; Cauchon, E., et al. 2009. Anal Biochem.

The term “host cell” refers to a cell that can be used to express a nucleic acid encoding a binding polypeptide of the present invention. A host cell can be a prokaryote, for example, E. coli, or it can be a eukaryote, for example, a single-celled eukaryote (e.g., a yeast or other fungus), a plant cell (e.g., a tobacco or tomato plant cell), an animal cell (e.g., a human cell, a monkey cell, a hamster cell, a rat cell, a mouse cell, or an insect cell) or a hybridoma. Examples of host cells include Chinese hamster ovary (CHO) cells or their derivatives such as Veggie CHO and related cell lines which grow in serum-free media (see Rasmussen et al., Cytotechnology 28: 31, 1998) or CHO strain DX-B11, which is deficient in DHFR (see Urlaub et al., Proc. Natl. Acad. Sci. USA 77: 4216-4220, 1980).

The term “human antibody” or “fully human antibody” refers to an antibody in which both the constant regions and the framework consist of fully or substantially human sequences such that the human antibody typically elicits substantially no immunogenic reaction against itself when administered to a human and, preferably, elicits no detectable immunogenic response. Thus, the defined terms contemplate minor amino acid modifications (often no more than 1, 2, 3, 4, or 5 amino acid substitutions, additions, or deletions) made relative to a native human antibody sequence to allow, for example, for improved formulation, stability, or manufacturability (e.g., removal of unpaired cysteine residues).

The term “humanized antibody” refers to an isolated antibody in which substantially all of the constant region is derived from or corresponds to human immunoglobulins, while all or part of one or more variable regions is derived from another species, for example a mouse.

The term “isolated” refers to a compound that: (1) is substantially purified (e.g., at least 60%, 70%, 80%, or 90%) away from cellular components with which it is admixed in its expressed state such that it is the predominant species present, (2) is conjugated to a polypeptide or polynucleotide or other moiety to which it is not linked in nature, (3) does not occur in nature as part of a larger polypeptide or polynucleotide sequence, (4) is combined with other chemical or biological agents having different specificities in a well-defined composition, or (5) comprises a human engineered sequence not otherwise found in nature.

The term “low-affinity” in reference to binding polypeptides, such as an Fc-polypeptide, means that they have no, or substantially no, specific binding to FCGR3A on mammalian NK cells such as human NK cells. In some embodiments, low-affinity results from removal, truncation, or modification (e.g., by aglycosylation) of the Fc of an Fc-polypeptide such that its capacity to specifically bind to FCGR3A is absent or substantially absent.

The term “mammal” specifically includes reference to at least one of a: human, chimpanzee, rhesus monkey, cynomologous monkey, dog, cat, mouse, or rat.

The terms “monoclonal antibody” or “monoclonal antibody composition” refers to a preparation of isolated antibody molecules of single molecular composition (notwithstanding minor heterogeneities resulting from, for example, post-translational modification such as glycosylation and/or signal sequence cleavage), typically encoded by the same nucleic acid molecule. A monoclonal antibody composition displays a single binding specificity and affinity for a particular epitope. In certain embodiments, monoclonal antibodies are produced by a single hybridoma or other cell line (e.g., a transfectoma), or from a transgenic mammal such as cloning from a transgenic B-cell. The term “monoclonal” is not limited to any particular method for making an antibody. In some embodiments the monoclonal antibodies are human monoclonal antibodies.

The term “naturally occurring” or “native” when used in connection with biological materials such as nucleic acid molecules, polypeptides, host cells, and the like, refers to those which are found in nature and not modified by human intervention.

The terms, “nucleic acid” and “polynucleotide” refer to a deoxyribonucleotide or ribonucleotide polymer, or chimeras thereof, and unless otherwise limited, encompasses the complementary strand of the referenced sequence. A nucleic acid sequence is “operably linked” to a regulatory sequence if the regulatory sequence affects the expression (e.g., the level, timing, or location of expression) of the nucleic sequence. A “regulatory sequence” is a nucleic acid that affects the expression (e.g., the level, timing, or location of expression) of a second nucleic acid. Thus, a regulatory sequence and a second sequence are operably linked if a functional linkage between the regulatory sequence and the second sequence is such that the regulatory sequence initiates and mediates transcription of the DNA sequence corresponding to the second sequence. Examples of regulatory sequences include promoters, enhancers and other expression control elements (e.g., polyadenylation signals). Further examples of regulatory sequences are described in, for example, Goeddel, 1990, Gene Expression Technology: Methods in Enzymology 185, Academic Press, San Diego, Calif. and Baron et al., Nucleic Acids Res. 23: 3605-3606, 1995.

The terms “peptide,” “polypeptide”, and “protein” are used interchangeably throughout and refer to a molecule comprising two or more amino acid residues joined to each other by peptide bonds. The terms “polypeptide”, “peptide”, and “protein” are also inclusive of modifications including, but not limited to, glycosylation, lipid attachment, sulfation, gamma-carboxylation of glutamic acid residues, hydroxylation, and ADP-ribosylation.

The term “peptibody” refers to a particular type of binding peptide, and more specifically, a type of Fc-polypeptide. In some embodiments the Fc of the peptibody is a human IgG1 or IgG2 Fc. The structure and production of peptibodies is generally described in PCT publication WO 00/24782, published May 4, 2000, incorporated herein by reference. Exemplary peptides may be generated by any of the methods set forth herein, such as carried in a peptide library (e.g., a phage display library), generated by chemical synthesis, derived by digestion of proteins, or generated using recombinant DNA techniques.

The term “peptibody fragment” or “antibody fragment” refers to a peptide of a peptibody or antibody which comprises less than a complete intact peptibody or antibody but retains the ability to specifically bind to its target molecule (i.e., human DR5 or human DR4). Exemplary fragments includes F(ab) or F(ab′)2 fragments. Such a fragment may arise, for example, from a truncation at the amino terminus, a truncation at the carboxy-terminus, and/or an internal deletion of a residue(s) from the amino acid sequence. Fragments may result from alternative RNA splicing or from in vivo or in vitro protease activity. Such fragments may also be constructed by chemical peptide synthesis methods, or by modifying a polynucleotide encoding an antibody or peptibody.

The terms “polynucleotide,” “oligonucleotide”, and “nucleic acid” are used interchangeably throughout and include DNA molecules (e.g., cDNA or genomic DNA), RNA molecules (e.g., mRNA), and hybrids thereof. The nucleic acid molecule can be single-stranded or double-stranded.

The term “specifically binds” refers to the ability of a binding polypeptide of the present invention, under specific binding conditions, to bind to a target (e.g., human DR5, human DR4, or human FCGR3A) such that its affinity is at least 10 times as great, but optionally 50 times as great, 100, 250 or 500 times as great, or even at least 1000 times as great as the average affinity of the same molecule to a collection of random peptides or polypeptides of sufficient statistical size. A binding polypeptide need not bind exclusively to a single target molecule but may specifically bind to a non-target molecule due to similarity in structural conformation between the target and non-target (e.g., paralogs or orthologs). Those of skill will recognize that specific binding to a molecule having the same function in a different species of animal (i.e., ortholog) or to a molecule having a substantially similar epitope as the target molecule (e.g., a paralog) is within the scope of the term “specific binding” which is determined relative to a statistically valid collection of unique non-targets (e.g., random polypeptides). Thus, an anti-DR5 binding polypeptide of the invention may specifically bind to more than one distinct species of target molecule, such as specifically binding (i.e., cross-reacting) to both DR5 and DR4. Solid-phase ELISA immunoassays can be used to determine specific binding. Generally, specific binding proceeds with an association constant of at least about 1×10⁷ M⁻¹, and often at least 1×10⁸ M⁻¹, 1×10⁹ M⁻¹, or 1×10¹⁰ M⁻¹.

The terms “synergy”, “synergistic”, “synergistically”, or “synergize” in the context of induction of apoptosis by a combination of therapeutic agents (i.e., an “apoptotic composition), such as the binding polypeptides of the invention, means that the activity of the combination in inducing cell death (e.g., via apoptosis) is greater than the additive effect of each agent. Additivity excess is the amount that the synergistic activity exceeds that of full additivity.

The term “TRAIL”, “tumor necrosis factor related apoptosis inducing ligand” or “Apo2 ligand” is a homotrimeric ligand that interacts with four members of the TNF-receptor superfamily (TRAIL receptors 1 to 4), as well as with the soluble osteoprotegerin (“OPG”) receptor. The term is inclusive of human TRAIL (see, U.S. Pat. Nos. 6,046,046; 6,284,236; 6,998,116, all of which are incorporated herein by reference), as well as homologs found in mammals generally, including mice, rats, and cynomologous monkeys. Agonistic (apoptosis inducing) TRAIL variants include multimeric forms of TRAIL (e.g., dimers, trimers), truncated versions of TRAIL such as LZ (leucine zipper)-TRAIL, or derivitized or recombinantly modified versions of TRAIL such that the resultant molecule retains agonistic activity with respect to apoptosis sensitive cells expressing DR4 and/or DR5 (e.g., Colo205). TRAIL and TRAIL variants are detailed in U.S. 20060141561 and U.S. Pat. No. 7,994,281, both of which are incorporated herein by reference.

The term “vector” refers to a nucleic acid used in the introduction of a polynucleotide of the present invention into a host cell. Vectors are often replicons. Expression vectors permit transcription of a nucleic acid inserted therein when present in a suitable host cell or under suitable in vitro conditions.

### Anti-DR5 Binding Polypeptides and Polynucleotides Encoding Same

The binding polypeptides of the present invention specifically bind, under specific binding conditions, to the extracellular domain of mammalian DR5 (anti-DR5 binding polypeptides) and/or DR4 (anti-DR4 binding polypeptides) expressed on a mammalian cell, such as a mammalian cancer cell. In some embodiments the DR5 and/or DR4 receptors to which the binding polypeptides specifically bind will be expressed on human, cynomologous monkey, rat, or mouse cancer cells. In some embodiments, the binding polypeptide will specifically bind to DR4 and/or DR5 receptors of two or more mammalian species. For example, a binding polypeptide of the invention can specifically bind to DR4 and/or DR5 of at least both human and cynomologous monkey, or at least human and murine DR4 and/or DR5 receptors. Cross-reactivity of binding polypeptides to two or more species of mammals is therefore included within the scope of the invention.

In some embodiments, a binding polypeptide of the invention is an agonist of the DR5 and/or DR4 receptor to which it's targeted. In other embodiments it is not an agonist of the targeted receptor. Thus, for example, while a first Fc-polypeptide alone may itself exhibit no detectable agonistic activity in vitro and/or in vivo towards a particular death receptor, in combination with TRAIL or a second Fc-polypeptide they will jointly cooperate and exhibit agonistic activity toward that DR5 and/or DR4 receptor in excess of that exhibited by TRAIL or the second Fc-polypeptide alone. In other embodiments, however, the first Fc-polypeptide will exhibit some degree of agonistic activity of its own in addition to cooperating with TRAIL or a second Fc-polypeptide. An anti-DR5 binding polypeptide can be demonstrated to be agonistic using well known in vivo and/or in vitro apoptosis assays with a DR5 expressing mammalian cell, such as but not limited to: Colo205 (colon), WM35 (colon), H1975 (lung), SK-MES-1 (lung), HCC38 (breast), H2122 (lung), SkLu1 (lung), or H460 (lung).

In some embodiments, the binding polypeptides of the invention are exogenous cross-linking dependent wherein they require the presence of an exogenous cross-linking agent, such as Protein G, to induce apoptosis in an apoptosis sensitive cell in vitro. Conversely, in some embodiments the binding polypeptides of the present invention are exogenous cross-linking independent wherein they induce apoptosis in apoptosis sensitive cells in vitro without an exogenous cross-linking agent. Methods of assaying for exogenous cross-linking dependence or independence of an anti-DR4 and/or anti-DR5 binding polypeptide are known in the art as are apoptosis sensitive DR4 and/or DR5 mammalian cell lines (supra). Exemplary exogenous cross-linking agents include secondary antibodies that specifically bind to the binding polypeptides wherein the secondary antibodies coat a solid support or are components of a liposomal membrane, or the use of Protein A or Protein G. Without being bound by theory, it is believed that clustering of the death receptors plays a role in the apoptotic induction mechanism. Thus, exogenous cross-linking agents act to cluster DR4 and/or DR5 via specific binding to, and clustering of, binding polypeptides.

In some embodiments the binding polypeptides of the present invention are Fc-polypeptides, such as antibodies or peptibodies. In some embodiments the Fc-polypeptides are human antibodies such as IgG1 or IgG2 antibodies. In some embodiments the Fc-polypeptides are bispecific Fc-polypeptides and, accordingly, specifically bind to two distinct sites on DR5 or DR4, or specifically bind to both DR4 and DR5. Fc-polypeptides can exhibit bispecificity to distinct sites on their target by virtue of comprising two distinct species (bivalent) of binding polypeptides each of which binds to a unique site. Alternatively, bispecificity of an Fc-polypeptide can result from cross-reactivity of a single species (monovalent) of binding polypeptide to two unique sites having sufficient similarity in structure. Such cross-reactive binding polypeptides can be made to specifically bind to regions on both DR4 and DR5 owing to substantial homology between these two death receptors.

Those of skill in the art will recognize that the Fc of an Fc-polypeptide, such as an antibody, can be modified to increase or reduce specific binding affinity to an FCGR3A receptor. Compositions and methods relating to high-affinity IgG1 Fc-polypeptides is discussed in the International Patent Application of Graves et al. PCT/US2011/036521, filed internationally on 13 May 2011, the entire contents of which is incorporated herein by reference. High-affinity Fc-polypeptides can comprise an afucosylated Fc to increase affinity to mammalian (e.g., human) FCGR3A. In some embodiments the Fc is an afucosylated fully human IgG1 Fc. In some embodiments the Fc-polypeptide is an afucosylated fully human IgG1 monoclonal antibody. In some embodiments the afucosylated fully human IgG1 monoclonal antibody specifically binds to human DR5 and/or human DR4. Thus in some embodiments the afucosylated fully human IgG1 monoclonal antibody is a bispecific antibody that specifically binds to human DR5 and human DR4. In some embodiments the Fc-polypeptide is a fully human IgG1 monoclonal antibody that specifically binds to human DR5 but does not specifically bind to (i.e., does not cross-react with) human DR4. In some embodiments the Fc-polypeptide specifically binds to human DR4 but does not specifically bind to (i.e., does not cross react with) human DR5. Methods of creating afucosylated antibodies or Fc-fusion peptides are known in the art and include, but are not limited to, recombinant expression using genetic (e.g., siRNA) or chemical means to inhibit cellular fucosyl transferase function or expression, using host cells missing the gene for fucosyl transferase (e.g., fut8 knock-outs), or defucosylating the Fc by in vitro chemical or enzymatic means. See, e.g., U.S. Pat. No. 6,946,292, incorporated herein by reference. Those of skill will recognize that compositions comprising a plurality of high-affinity Fc-polypeptides of the invention need not be 100% afucosylated to exhibit enhanced anti-cancer activity but generally comprise at least 40%, 50%, 60%, 70%, 75%, 80%, 85%, 90%, 95%, 97%, 98%, or 99% afucosylated Fc-polypeptides.

In another approach the present invention provides an agonistic high-affinity Fc-polypeptide wherein the Fc comprises at least one amino acid substitution that yields enhanced FCGR3A affinity as described in U.S. Pat. No. 7,317,091 (incorporated herein by reference). In some embodiments, the Fc comprising an aforementioned amino acid substitution is a human IgG1 Fc. In some embodiments the Fc-polypeptide comprising at least one amino acid substitutions to enhance FCGR3A binding is a fully human IgG1 monoclonal antibody. In some embodiments the fully human IgG1 monoclonal antibody specifically binds to human DR5 and/or human DR4. Thus in some embodiments the fully human IgG1 monoclonal antibody is a bispecific antibody that specifically binds to human DR5 and human DR4. In some embodiments the Fc-polypeptide is a fully human IgG1 monoclonal antibody that specifically binds to human DR5 but does not specifically bind to (i.e., does not cross-react with) human DR4. In some embodiments the Fc-polypeptide specifically binds to human DR4 but does not specifically bind to (i.e., does not cross react with) human DR5. In some embodiments, the Fc comprises a substitution at, at least one of, residues: 230, 233, 234, 235, 239, 240, 243, 264, 266, 272, 274, 275, 276, 278, 302, 318, 324, 325, 326, 328, 330, 332, and 335, wherein the numbering of the residues in the Fc region is that of the EU index as in Kabat. In some embodiments, the Fc comprises at least one amino acid substitution selected from the group consisting of: P230A, E233D, L234E, L234Y, L234I, L235D, L235S, L235Y, L2351, S239D, S239E, S239N, S239Q, S239T, V2401, V240M, F243L, V2641, V264T, V264Y, V266I, E272Y, K274T, K274E, K274R, K274L, K274Y, F275W, N276L, Y278T, V302I, E318R, S324D, S324I, S324V, N325T, K326I, K326T, L328M, L328I, L328Q, L328D, L328V, L328T, A330Y, A330L, A330I, I332D, I332E, I332N, I332Q, T335D, T335R, and T335Y wherein the letter preceding the number represents in one-letter amino acid code the substitution residue, the number indicates the residue of the Fc numbered per the EU index as in Kabat and the letter following the number indicates the native residue. In some embodiments, the high-affinity Fc-polypeptide comprises both an afucosylated Fc and an amino acid substitution to enhance FCGR3A affinity as described above. In some embodiments, the Fc of the Fc-polypeptide comprises 1, 2, 3, 4, 5, 6, 7, 8, 9, or 10 of the substitutions to increase affinity to FCGR3A.

The Fc of an Fc-polypeptide can be modified to reduce specific binding affinity to the FCGR3A receptor. Low-affinity Fc-polypeptides have a reduced ability to be cross-linked by FCGR3A bearing cells, such as NK cells or macrophages, and therefore low-affinity Fc-polypeptides that specifically bind to DR5 and/or DR4 can be constructed and utilized to reduce or eliminate NK or macrophage mediated apoptosis. Methods of reducing or substantially eliminating specific binding of an Fc to FCGR3A are known in the art. Low-affinity Fc-polypeptides can be obtained by constructing or modifying the Fc of the Fc-polypeptide by a variety of means such as by providing a human IgG1 Fc comprising an N297A aglycosylation mutation, use of an IgG2 Fc, use of an Fc which is enzymatically and/or chemically aglycosylated to yield low-affinity Fc-polypeptides, or use of Fc-polypeptides that are made from host cells or in culture media in which glycosylation is inhibited or prevented so as to yield low-affinity Fc-polypeptides. Thus, in one aspect the present invention provides an aglycosylated Fc of the Fc-polypeptide. In some embodiments, the Fc of the Fc-polypeptide is an IgG2 Fc. In some embodiments, the IgG1 or IgG2 Fc is a human Fc. In some embodiments, the Fc of the Fc-polypeptide comprises an N297A substitution resulting in aglycosylation at that position. See, Sazinsky, et al., PNAS 105: 20167-20172 (2008). In certain embodiments, the Fc comprising the N297A substitution is a human IgG1 Fc. In some embodiments, the present invention comprises Fc-polypeptides in which the Fc is sufficiently truncated such that is unable to specifically bind to FCGR3A.

Methods of identifying, isolating, or otherwise making Fc-polypeptides of the present invention are known to those of ordinary skill in the art. The polypeptide component of an Fc-polypeptide of the invention can be obtained from a number of sources such as a phage or polypeptide library. Methods of making and screening phage or polypeptide libraries are well known in the art. Phage and polypeptide libraries are also available commercially. Polypeptides having the desired specific binding properties can be covalently attached, directly or indirectly (i.e., via a linker), to an Fc to yield the Fc-polypeptide. In some embodiments the Fc-polypeptide is a bivalent IgG1 antibody, such as a fully human monoclonal antibody. In some embodiments, the polypeptide of the Fc-polypeptide is a scFv (single-chain Fv), Fab or F(ab′)₂ fragment of an antibody. Representative Fc-polypeptides that can be modified according to the methods of the invention to yield a high- or low-affinity Fc-polypeptide include the anti-DR5 antibody conatumumab (Amgen Inc.). In one embodiment, the Fc-polypeptide is a bispecific multivalent Fc-polypeptide comprising human TRAIL (TNF-Receptor Apoptosis Inducing Ligand) and an anti-DR5 and/or DR4 peptide.

The Fc of an Fc-polypeptide of the invention can be obtained by a variety of methods well known in the art including, but not limited to, recombinant expression methods, solid-phase peptide synthetic methods, isolated from natural sources such as human immunoglobulins, or combinations of these methods. In some embodiments, the Fc is a human IgG1. In certain embodiments, the Fc of one isotype is converted to a different isotype by isotype switching. Methods of isotype switching include, but are not limited to, direct recombinant techniques and cell-cell fusion techniques (see e.g., U.S. Pat. No. 5,916,771), among others. In certain embodiments, an Fc is converted from a human IgG2, IgG3, or IgG4 subclass to a human IgG1 subclass. Those of skill in the art will recognize that in order to optimize solubility, manufacturability, stability, and other factors relevant to the manufacture of biopharmaceuticals, several amino acid residues of a native human IgG1 can be modified yet still be within the definition of human IgG1. Generally, no more than a total of up to 15 residues are deleted, added, and/or substituted and often no more than 10, 9, 8, 7, 6, 5, 4, 3, 2, or 1. The Fc of an Fc-polypeptide can, however, be linked directly or indirectly with labels, toxins, drugs, tissue-specific binding agents, and the like, to enhance the pharmacokinetic or pharmacodynamic properties of the Fc-polypeptide.

In some embodiments, two or more Fc-polypeptides can be covalently bonded to one another, such as by cysteine-cysteine disulfide bonds, to create bivalent (i.e., two antigen binding sites), trivalent, or higher order structures of Fc-polypeptides. A bivalent Fc-polypeptide, such as an antibody, can be monospecific and specifically bind to a single epitope of the target, or bispecific such that it specifically binds to two unique epitopes on the same target (e.g., human DR5 or human DR4) or two unique epitopes of differing targets (e.g., human DR4 and human DR5). In additional embodiments, two or more unique or identical species of polypeptides that specifically bind to human DR4 and/or human DR5 are covalently linked to a single Fc to form an Fc-polypeptide. Thus, in some embodiments, 2, 3, or 4 of such polypeptides are covalently linked to a single Fc. Such Fc-polypeptides can be dimerized (by, for example, disulfide bonding between Fc chains to form a bivalent Fc-polypeptide), trimerized, etc. The polypeptide can be directly or indirectly attached to an Fc at or near the N- or C-terminus of the Fc or at an internal residue within the Fc. In other embodiments, a second or additional polypeptide that specifically binds to human DR4 and/or human DR5 is covalently linked to a polypeptide that itself is covalently linked to the Fc. Thus, Fc-polypeptides can comprise multiple polypeptides covalently linked, directly or indirectly, to the Fc or to a polypeptide that is itself covalently attached directly or indirectly to the Fc.

In certain embodiments, a binding polypeptide of the invention can be constructed using recombinant methods. Therefore, another aspect of the invention is a polynucleotide encoding a binding polypeptide of the invention or component elements of a binding polypeptide of the present invention. Component elements of a binding polypeptide can be fully assembled to form a binding polypeptide by standard chemical, biochemical, or recombinant methods. In another aspect the present invention comprises an expression vector comprising the polynucleotide encoding a binding polypeptide. In certain embodiments, the expression vectors comprise control sequences (e.g., promoters, enhancers) that are operably linked to a polynucleotide encoding the binding polypeptide so as to support expression in a suitable host cell. In certain embodiments, the expression vector also comprises polynucleotide sequences that allow chromosome-independent replication in the host cell. Exemplary vectors include, but are not limited to, plasmids, cosmids, and YACS. In yet another aspect, the invention comprises a host cell comprising the expression vector of the invention. Methods of transfecting suitable host cells (e.g., CHO cells) with the expression vector of the invention and culturing the transfected host cells under conditions suitable for expression of an Fc-polypeptide are known in the art. The transfection procedure used may depend upon the host to be transformed. Certain methods for introduction of heterologous polynucleotides into mammalian cells are known in the art and include, but are not limited to, dextran-mediated transfection, calcium phosphate precipitation, polybrene mediated transfection, protoplast fusion, electroporation, encapsulation of the polynucleotide(s) in liposomes, and direct microinjection of the DNA into nuclei. Certain mammalian cell lines available as hosts for expression are known in the art and include, but are not limited to, many immortalized cell lines available from the American Type Culture Collection (ATCC), including but not limited to Chinese hamster ovary (CHO) cells, E5 cells, baby hamster kidney (BHK) cells, monkey kidney cells (COS), human hepatocellular carcinoma cells (e.g., Hep G2), and a number of other cell lines. In certain embodiments, cell lines may be selected through determining which cell lines have high expression levels and produce Fc-polypeptides with desired antigen binding properties. Binding polypeptides that are Fc-polypeptides, and specifically antibodies, can be obtained using any of a variety of methods for antibody production. Methods of making monoclonal antibodies, such as human monoclonal antibodies, are well known in the art.

### Apoptotic Compositions

The present invention provides binding polypeptide of the present invention in compositions (“apoptotic compositions”) that act cooperatively in vitro and/or in vivo to induce apoptosis in a mammalian cancer cell expressing DR5 and/or DR4. In some embodiments, the mammalian cancer cell is a human, murine, or cynomologous monkey cancer cell. In some embodiments, the apoptotic composition comprises at least a first anti-DR5 and/or anti-DR4 binding polypeptide of the present invention and at least a second binding polypeptide that specifically binds, under specific binding conditions, to at least the same receptor as the first binding polypeptide. In this embodiment, the first and second binding polypeptides specifically bind, under specific binding conditions, to the extracellular domain of the target receptor (DR5 and/or DR4). The first binding polypeptide and second binding polypeptide of the apoptotic composition do not substantially competitively inhibit each other from specifically binding to the target receptor so as to allow for cooperativity. Instrumentation and methods of assaying and quantifying competitive inhibition, such as by competitive ELISA, are known in the art such as the AlphaLISA™ system (Perkin Elmer, Mass. USA) ELISA assay. Generally, while some degree of competitive inhibition is acceptable, it is preferably minimized. Thus, the specific binding of a first binding polypeptide should not reduce the specific binding of a second binding polypeptide (or vice-versa) by more than 30%, 40%, 50%, 60%, 70%, 80%, 90%, or 95%. In some embodiments of the apoptotic compositions of the present invention, at least 1, 2, 3, 4, or more of the binding polypeptides are agonistic as single agents towards the DR5 and/or DR4 expressing apoptosis sensitive mammalian cancer cells. Examples of such DR4 and/or DR5 expressing apoptosis sensitive mammalian cancer cells are discussed above and in the working examples where a number of cell lines are disclosed. In some embodiments, specific binding of the first binding polypeptide and the second binding polypeptide cooperatively induce apoptosis of the mammalian cancer cell independent of natural killer (NK) cell mediation of apoptosis. Accordingly, in some embodiments the first and/or second binding polypeptides are low-affinity (for FCGR3A) binding polypeptides, such as low-affinity Fc-polypeptides, such as but not limited to, low-affinity antibodies. While some degree of NK mediated apoptosis is permissible, NK-dependent apoptotic activity of the apoptotic composition is often less than 10%, and in some embodiments less than 5%, 3%, 2%, 1% or 0.5% of the total apoptotic activity (i.e., NK-independent+NK-dependent apoptosis). Binding polypeptides having a low-affinity for FCGR3A can lack or have a truncated Fc such that the binding polypeptide lacks the ability to bind to FCGR3A. In some embodiments a binding polypeptide of the present invention is an IgG2 Fc-polypeptide. In specific embodiments, the first and second binding polypeptides are Fc-polypeptides such as antibodies. In even more specific embodiments, the antibodies are human antibodies that specifically bind to human DR5 and/or DR4. The first and second anti-DR5 binding polypeptides can specifically bind to the same receptor (DR5 or DR4). An apoptotic composition can be a single bispecific molecule comprising a first and second binding polypeptide of the invention. In one embodiment, the single molecule is a bispecific anti-DR5 human or humanized antibody. In some embodiments, the apoptotic composition comprises conatumumab (AMG 655; Amgen Inc.) whose structure is provided in U.S. Pat. No. 7,521,048 of Gliniak et al, filed, Aug. 28, 2006, incorporated by reference herein, and disclosed therein as antibody “O” (SEQ ID NO: 30 and 64). While apoptotic compositions often comprise two binding polypeptides of the invention, 3, 4, 5, or more can be utilized. In some embodiments, the binding polypeptides of the invention are conjugated to each other directly or indirectly. Thus, a single molecule can comprise multiple species of binding polypeptide.

In another embodiment, an apoptotic composition comprises at least a first anti-DR5 and/or anti-DR4 binding polypeptide of the present invention and at least one tumor necrosis factor related apoptosis inducing ligand (TRAIL) or TRAIL variant, such as LZ-TRAIL, that cooperate to induce apoptosis in apoptosis sensitive DR4 and/or DR5 expressing cancer cells in vivo and/or in vitro. In this embodiment, the first binding polypeptide and TRAIL do not substantially competitively inhibit each other from specifically binding to the extracellular domain of the target death receptor (DR4 and/or DR5). Generally, while some degree of competitive inhibition is acceptable, it should be minimized so as to not adversely impede mutual specific binding of the binding polypeptide and TRAIL or TRAIL variant. Thus, the specific binding of a first binding polypeptide (or TRAIL or TRAIL variant) should not reduce the specific binding of TRAIL or TRAIL variant (or the first binding polypeptide) by more than 30%, 40%, 50%, 60%, 70%, 80%, 90%, or 95%. In some embodiments, the apoptotic composition cooperatively induces apoptosis of the mammalian cancer cell independent of natural killer (NK) cell mediation of apoptosis. Accordingly, in some embodiments the first binding polypeptide is a low-affinity (for FCGR3A) binding polypeptide, such as a low-affinity Fc-polypeptide, often a low-affinity antibody. Thus, in some embodiments the NK-dependent apoptotic activity of the binding polypeptides of the invention is less than 10%, and in some embodiments less than 5%, 3%, 2%, 1% or 0.5% of the total apoptotic activity (i.e., NK-independent+NK-dependent apoptosis). In some embodiments, at least 1, 2, 3, 4, or more of the binding polypeptides of the apoptotic composition are agonistic as single agents towards DR5 and/or DR4 expressing apoptosis sensitive mammalian cancer cells. In some embodiments, the mammalian cancer cell is a human cancer cell. In some embodiments, the first binding polypeptide is not agonistic towards DR5 and/or DR4 expressing apoptosis sensitive cancer cells. Examples of such DR4 and/or DR5 expressing apoptosis sensitive mammalian cancer cells are discussed above and in the working examples where a number of cell lines are disclosed. In specific embodiments, the first binding polypeptides are Fc-polypeptides such as antibodies. In even more specific embodiments, the antibodies are human antibodies. In a specific embodiment, the apoptotic composition comprises human TRAIL and conatumumab (AMG 655).

The present invention also provides a method to identify and isolate binding polypeptides that cooperatively induce apoptosis, of a DR5 and/or DR4 expressing apoptosis sensitive mammalian cancer cell, in combination with each other or with TRAIL or a TRAIL variant, such as LZ-TRAIL. Apoptosis sensitive tumor cell lines that can be used to identify apoptotic compositions of the invention are known in the art and include, but not limited to, Colo205 (colon), WM35 (colon), H1975 (lung), SK-MES-1 (lung), HCC38 (breast), H2122 (lung), SkLu1 (lung), or H460 (lung). Apoptosis assays for evaluating the efficacy and/or potency of apoptotic compositions are known in the art. See, for example, caspase activation and cell viability assays in Kaplan-Lefko et. al., Cancer Biology & Therapy, 9:8, 1-14, (2010). Conveniently, a two-dimensional or higher order matrix of the binding polypeptides being evaluated for their ability to exhibit cooperativity can be created to efficiently assess candidate binding polypeptides. In a two-dimensional matrix, for example, large numbers of binding polypeptides (or binding polypeptides and TRAIL or TRAIL variant) can be arrayed in each dimension and pairwise combinations that exhibit cooperativity can thereby be efficiently and readily identified. In some embodiments, the anti-DR5 and/or anti-DR4 binding polypeptides utilized in the assay for apoptotic cooperativity are pre-selected on the basis of desired characteristics to improve the frequency of cooperative pairings. Thus, in some embodiments, the binding polypeptides (or the binding polypeptide and TRAIL or TRAIL variant) utilized in the assay do not competitively inhibit mutual specific binding to their target (i.e., DR4 and/or DR5) as discussed in more detail supra. In some embodiments, at least one of the binding polypeptides utilized in the assay and in the apoptotic composition is an agonistic binding polypeptide to DR4 and/or DR5. In some embodiments, the binding polypeptides utilized in the screening are Fc-polypeptides, such as antibodies. In some embodiments, the antibodies utilized in the screen will require a cross-linker, such as Protein G, to induce apoptosis of a sensitive cancer cell in vivo. In some embodiments, these antibodies are human monoclonal antibodies. In some embodiments, one of the cross-linking dependent antibodies is conatumumab (AMG 655; Amgen Inc).

### Therapeutic Applications

An apoptotic composition of the invention can be used as a “therapeutic composition” to inhibit growth of mammalian, particulary human, cancer cells as a monotherapy (i.e., as a single agent), in combination with at least one chemotherapeutic agent (i.e., a combination therapy), and/or in combination with radiation therapy. An effective amount of a therapeutic composition is administered to inhibit, halt, or reverse progression of cancers that are sensitive to DR4 and/or DR5 mediated apoptosis. Human cancer cells can be treated in vivo, or ex vivo. In ex vivo treatment of a human patient, tissue or fluids containing cancer cells are treated outside the body and then the tissue or fluids are reintroduced back into the patient. In some embodiments, the cancer is treated in a human patient in vivo by administration of the therapeutic composition into the patient. Thus, the present invention provides ex vivo and in vivo methods to inhibit, halt, or reverse progression of the tumor, or otherwise result in a statistically significant increase in progression-free survival (i.e., the length of time during and after treatment in which a patient is living with pancreatic cancer that does not get worse), or overall survival (also called “survival rate”; i.e., the percentage of people in a study or treatment group who are alive for a certain period of time after they were diagnosed with or treated for cancer) relative to treatment with a control.

The cancers which can be treated by the methods of the invention include, but are not limited to, liver cancer, brain cancer, renal cancer, breast cancer, pancreatic cancer (adenocarcinoma), colorectal cancer, lung cancer (small cell lung cancer and non-small-cell lung cancer), spleen cancer, cancer of the thymus or blood cells (i.e., leukemia), prostate cancer, testicular cancer, ovarian cancer, uterine cancer, gastric carcinoma, head and neck squamous cell carcinoma, melanoma, and lymphoma. In some embodiments the cancer is non-small cell lung cancer (NSCLC).

### Pharmaceutical Compositions

The therapeutic compositions of the invention can each be administered alone as a monotherapy or as a combination therapy, i.e., combined with other agents (e.g., anti-angiogenic agents, chemotherapeutic agents, radiation therapy). Exemplary chemotherapeutic agents include, but are not limited to, adriamycin, doxorubicin, 5-fluorouracil, cytosine arabinoside, cyclophosphamide, thiotepa, docetaxel, busulfan, cytoxin, taxol, paclitaxel, methotrexate, gemcitabine, cisplatin, melphalan, vinblastine, bleomycin, etoposide, ifosfamide, mitomycin C, mitoxantrone, vincristine, vinorelbine, carboplatin, teniposide, daunomycin, carminomycin, aminopterin, dactinomycin, mitomycins, esperamicins, melphalan and other related nitrogen mustards.

A chemotherapeutic agent of the present invention can be administered prior to and/or subsequent to (collectively, “sequential treatment”), and/or simultaneously with (“concurrent treatment”) a specific binding agent of the present invention. Sequential treatment (such as pretreatment, post-treatment, or overlapping treatment) of the combination, also includes regimens in which the drugs are alternated, or wherein one component is administered long-term and the other(s) are administered intermittently. Components of the combination may be administered in the same or in separate compositions, and by the same or different routes of administration.

Exemplary cancer therapies, which may be co-administered with a therapeutic composition of the invention include, HERCEPTIN™ (trastuzumab), which may be used to treat breast cancer and other forms of cancer; RITUXAN™ (rituximab), ZEVALIN™ (ibritumomab tiuxetan), and LYMPHOCIDE™ (epratuzumab), which may be used to treat non-Hodgkin's lymphoma and other forms of cancer; GLEEVEC™ (imatinib mesylate), which may be used to treat chronic myeloid leukemia and gastrointestinal stromal tumors; and BEXXAR™ (tositumomab), which may be used for treatment of non-Hodgkin's lymphoma. Certain exemplary antibodies also include ERBITUX™; VECTIBIX™, IMC-C225; IRESSA™ (gefitinib); TARCEVA™ (ertinolib); KDR (kinase domain receptor) inhibitors; anti VEGF antibodies and antagonists (e.g., AVASTIN™ and VEGF-traps); anti-VEGF (vascular endothelial growth factor) receptor antibodies, peptibodies, and antigen binding regions; anti-Ang-1 and Ang-2 antibodies, peptibodies (e.g., AMG 386, Amgen Inc), and antigen binding regions; antibodies to Tie-2 and other Ang-1 and Ang-2 receptors; Tie-2 ligands; antibodies against Tie-2 kinase inhibitors; and CAMPATH™, (alemtuzumab).

### Pharmaceutical Formulation

A pharmaceutical composition comprising a therapeutic composition of the present invention may contain formulation materials for modifying, maintaining or preserving, for example, the pH, osmolarity, viscosity, clarity, color, isotonicity, odor, sterility, stability, rate of dissolution or release, adsorption, or penetration of the composition. The primary vehicle or carrier in a pharmaceutical composition may be either aqueous or non-aqueous in nature. For example, a suitable vehicle or carrier may be water for injection or physiological saline, possibly supplemented with other materials common in compositions for parenteral administration. Neutral buffered saline or saline mixed with serum albumin are further exemplary vehicles. Other exemplary pharmaceutical compositions comprise Tris buffer of about pH 7.0-8.5, or acetate buffer of about pH 4.0-5.5, which may further include sorbitol or a suitable substitute therefore. In one embodiment of the present invention, binding agent compositions may be prepared for storage by mixing the selected composition having the desired degree of purity with optional formulation agents (Remington's Pharmaceutical Sciences, supra) in the form of a lyophilized cake or an aqueous solution. Further, the binding agent product may be formulated as a lyophilizate using appropriate excipients such as sucrose.

The formulation components are present in concentrations that are acceptable to the site of administration. For example, buffers are used to maintain the composition at physiological pH or at slightly lower pH, typically within a pH range of from about 5 to about 8. A particularly suitable vehicle for parenteral administration is sterile distilled water in which a binding agent is formulated as a sterile, isotonic solution, properly preserved. Yet another preparation can involve the formulation of the desired molecule with an agent, such as injectable microspheres, bio-erodible particles, polymeric compounds (polylactic acid, polyglycolic acid), beads, or liposomes, that provide for the controlled or sustained release of the product which may then be delivered via a depot injection.

In another aspect, pharmaceutical formulations suitable for parenteral administration may be formulated in aqueous solutions, preferably in physiologically compatible buffers such as Hanks' solution, Ringer's solution, or physiologically buffered saline. Aqueous injection suspensions may contain substances that increase the viscosity of the suspension, such as sodium carboxymethyl cellulose, sorbitol, or dextran. Additional pharmaceutical compositions will be evident to those skilled in the art, including formulations involving binding agent molecules in sustained- or controlled-delivery formulations. Techniques for formulating a variety of other sustained- or controlled-delivery means, such as liposome carriers, bio-erodible microparticles or porous beads and depot injections, are also known to those skilled in the art. The pharmaceutical composition to be used for in vivo administration typically must be sterile. This may be accomplished by filtration through sterile filtration membranes. Where the composition is lyophilized, sterilization using this method may be conducted either prior to or following lyophilization and reconstitution. The composition for parenteral administration may be stored in lyophilized form or in solution. In addition, parenteral compositions generally are placed into a container having a sterile access port, for example, an intravenous solution bag or vial having a stopper pierceable by a hypodermic injection needle.

Once the pharmaceutical composition has been formulated, it may be stored in sterile vials as a solution, suspension, gel, emulsion, solid, or a dehydrated or lyophilized powder. Such formulations may be stored either in a ready-to-use form or in a form (e.g., lyophilized) requiring reconstitution prior to administration. An effective amount of a pharmaceutical composition to be employed therapeutically will depend, for example, upon the therapeutic context and objectives. One skilled in the art will appreciate that the appropriate dosage levels for treatment will thus vary depending, in part, upon the molecule delivered, the indication for which the binding agent molecule is being used, the route of administration, and the size (body weight, body surface or organ size) and condition (the age and general health) of the patient. Accordingly, the clinician may titer the dosage and modify the route of administration to obtain the optimal therapeutic effect. A typical dosage may range from about 0.1 mg/kg to up to about 50 mg/kg or more, depending on the factors mentioned above. In some embodiments, the dosage can be 1, 3, 5, 10, 15, 20, 25, or 30 mg/kg.

For any compound, the therapeutically effective dose can be estimated initially either in cell culture assays or in animal models such as mice, rats, rabbits, dogs, pigs, or monkeys. An animal model may also be used to determine the appropriate concentration range and route of administration. Such information can then be used to determine useful doses and routes for administration in humans. The exact dosage will be determined in light of factors related to the subject requiring treatment. Dosage and administration are adjusted to provide sufficient levels of the active compound or to maintain the desired effect. Factors that may be taken into account include the severity of the disease state, the general health of the subject, the age, weight, and gender of the subject, time and frequency of administration, drug combination(s), reaction sensitivities, and response to therapy. Long-acting pharmaceutical compositions may be administered every 3 to 4 days, every week, or biweekly depending on the half-life and clearance rate of the particular formulation. The frequency of dosing will depend upon the pharmacokinetic parameters of the molecule in the formulation used. Typically, a composition is administered until a dosage is reached that achieves the desired effect. The composition may therefore be administered as a single dose, or as multiple doses (at the same or different concentrations/dosages) over time, or as a continuous infusion. Further refinement of the appropriate dosage is routinely made. Appropriate dosages may be ascertained through use of appropriate dose-response data.

### EXAMPLES

Unless otherwise indicated, all cell lines were from the American Type Culture Collection (ATCC) or Amgen cell banks. Clinical grade AMG 655 was used for all in vitro and in vivo studies. All in vitro and in vivo experiments utilized untagged human TRAIL (amino acids 114-281) made at Amgen for research purposes. The polypeptide and polynucleotide sequences of the following antibodies disclosed in the application can be found in U.S. Pat. No. 7,521,048 (WO 2007/027713) and the designation of such antibodies are indicated parenthetically. The polypeptide and polynucleotide sequences of the following antibodies, and their respective CDRs (complementarity determining regions), are incorporated herein by reference. AMG 655=XG1-048=(Antibody O); 1.10.3=(Antibody G); 2.57.3=(Antibody H); 2.32.1=(Antibody L); 2.1.3=(Antibody D); 2.13.2=(Antibody C); 2.3.3=(Antibody I); 2.32.2=(Antibody J); 2.8.3=(Antibody E); 2.35.2=(Antibody A). The variable heavy chain of antibody 2.67.1 is provided at SEQ ID NO: 1 (DNA) and SEQ ID NO: 2 (polypeptide); the variable light chain for antibody 2.67.1 is provided at SEQ ID NO: 3 (DNA) and SEQ ID NO:4 (polypeptide). The variable heavy chain of antibody 2.11.1 is provided at SEQ ID NO: 5 (DNA) and SEQ ID NO: 6 (polypeptide); the variable light chain for antibody 2.11.1 is provided at SEQ ID NO: 7 (DNA) and SEQ ID NO: 8 (polypeptide).

### Example 1—Demonstrating Cooperativity in a Tumor Xenograft Model Using an Antibody Unable to Bind to Fc₁R (Fc-Gamma Receptors)

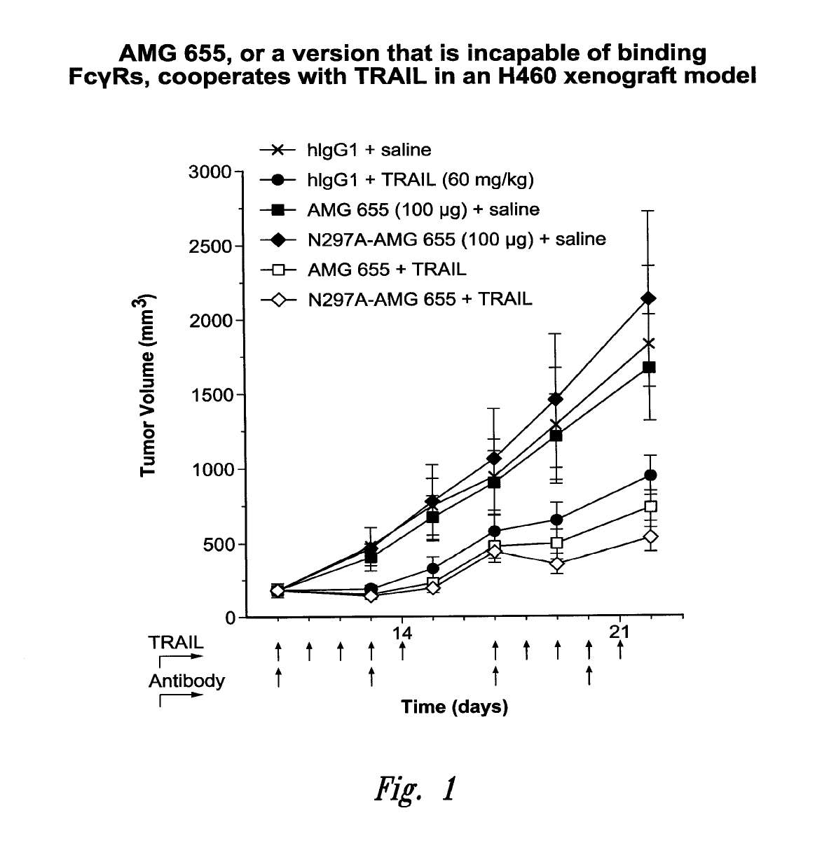

In order to determine whether cooperativity could be observed between TRAIL and the agonistic anti-DR5 antibody AMG 655 in vivo, TRAIL, AMG 655 and the two in combination were tested in an H460 lung cancer xenograft model. In addition, an aglycosylated mutant of AMG 655 (N297A) was included that is unable to bind FcγRs and therefore lacks intrinsic activity because it is unable to be cross-linked in vivo. Previous in vivo studies with H460 have shown that this tumor is sensitive to TRAIL but almost completely resistant to AMG 655. The efficacy of AMG 655 or AMG 655 N297A in the presence or absence of TRAIL was compared. For this experiment, CB17-SCID mice approximately 6 weeks of age were injected subcutaneously (s.c.) in the right flank with 5×10⁶ NCI-H460 cells. When tumors reached 150-200 mm³, mice were treated with either hIgG1 (human IgG1), AMG 655 (100 μg), AMG 655-N297A (100 μg), TRAIL (60 mg/kg) or a combination of AMG 655 and TRAIL, or AMG 655-N297A and TRAIL (n=10 mice/group). All antibodies were administered via intraperitoneal injection (i.p.) twice per week for 2 weeks. TRAIL and saline control were administered i.p. daily for 5 days for 2 weeks. Tumor dimensions were measured 2-3×/week by a digital caliper and tumor volumes were calculated as length×(width)²/2. FIG. 1 shows that whereas TRAIL induced significant and substantial tumor growth inhibition, no anti-tumor efficacy was observed with either AMG 655 or AMG 655-N297A alone. However, tumor growth inhibition in both combination treatment groups was significantly greater than that seen with TRAIL alone (p=0.001). These results indicate that both AMG 655 and AMG 655-N297A can cooperate with TRAIL in vivo and that this cooperativity is independent of FcγR-mediated cross-linking.

### Example 2—AMG 655 Potentiates Trail and LZ-Trail Mediated Killing of WM35 Cells in the Absence of Exogenous Cross-Linking

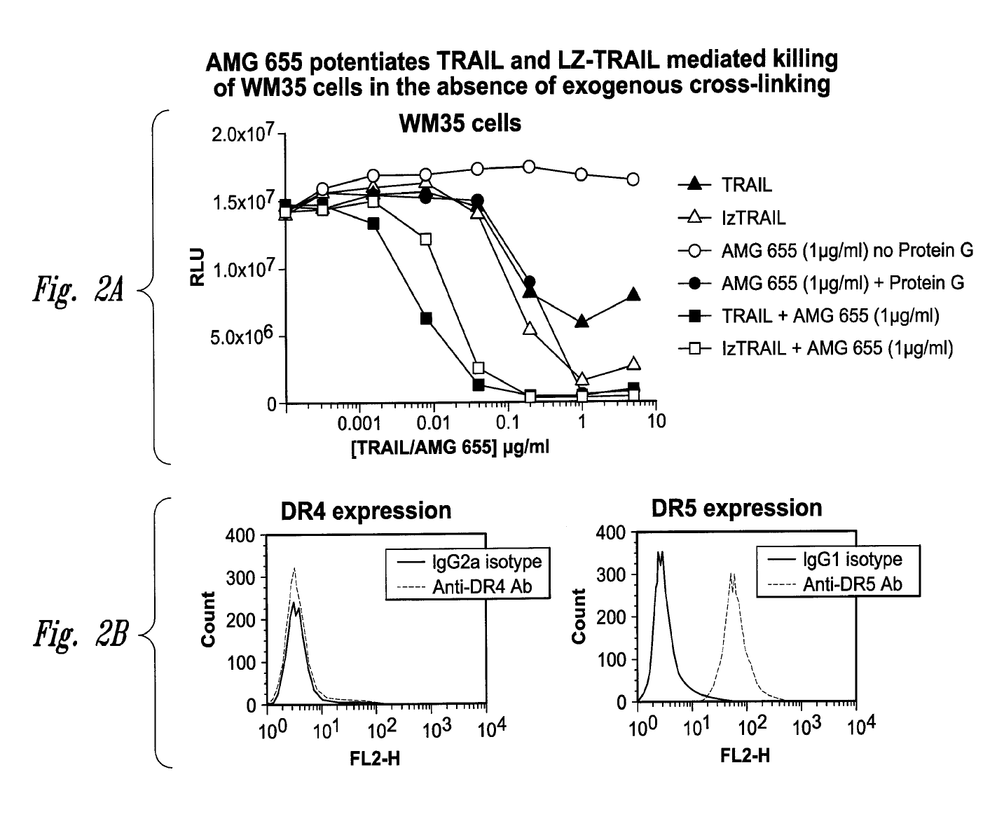

To study the relationship between TRAIL and AMG 655 binding to DR5, the effects in WM35 cells were evaluated. WM35 cells express surface DR5 but undetectable levels of DR4 (FIG. 2B), ensuring that TRAIL could only signal through DR5 and not DR4. In this experiment, 2×10\hat{ }4 WM35 cells/well were plated. The following day, the cells were treated with either media, hlgG (1 μg/mL) or AMG 655 (1 μg/mL). Cells were then treated with a dose titration of either AMG 655, TRAIL, or LZ-TRAIL as indicated (FIG. 2A). Protein G (1 μg/mL) was added as indicated. After a further 24 hour incubation, cell viability was determined using the CellGlo® system (Promega, USA). Data is expressed as relative luminescence units. This experiment was repeated at least 3 times with similar results. While LZ-TRAIL is an extremely potent version of TRAIL that is oligomerized by addition of a leucine zipper domain, the combination of TRAIL+AMG 655 was more potent than LZ-TRAIL for killing WM35 cells (FIG. 2A). Surface DR4 and DR5 expression levels on WM35 cells were analyzed by flow cytometry (FIG. 2B). Horizontal and vertical axes indicate the fluorescence intensity and relative number of cells, respectively. Solid black-lined histograms are isotype controls. Hatched-line histograms are antibodies against DR4 (M271 Ab) and DR5 (M413 Ab).

### Example 3—Cooperativity in Multiple Tumor Cell Lines

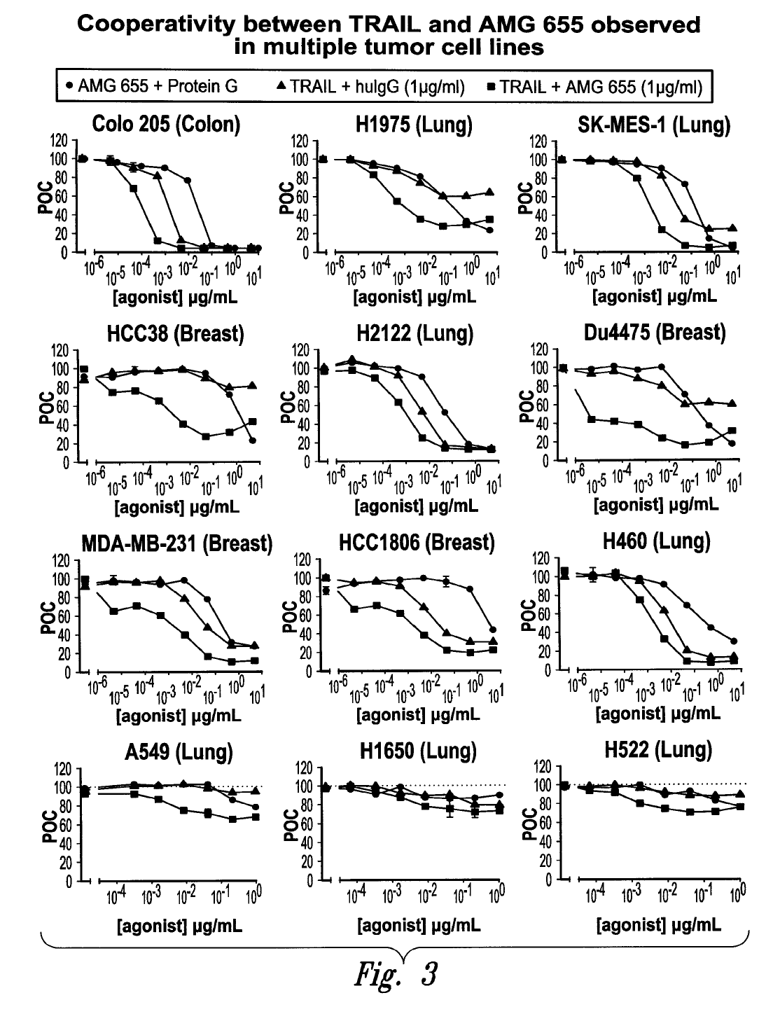

Whether synergy between TRAIL and AMG 655 could be observed in multiple tumor cell lines was addressed. Since most cell lines express both DR5 and DR4, a component of the apoptotic signal generated by TRAIL in the presence or absence of AMG 655 will be through DR4. Cells tested included a range of lung, colon and breast lines. These cell lines vary in their responsiveness to single agent TRAIL and AMG 655 (+the exogenous cross-linker protein G). For all the cell lines depicted, 2×10\hat{ }4 cells/well were plated. The following day, the cells were treated with media, hIgG (1 μg/mL) or AMG 655 (1 μg/mL). Cells were then treated with a dose titration of TRAIL or AMG 655 with protein G (1 μg/mL) as indicated (FIG. 3). After a further 24 hour incubation, cell viability was determined using the CellGlo® assay. Data are expressed as percent of control untreated cells. This experiment was repeated at least 3 times with similar results. The results in FIG. 3 show that cooperativity between TRAIL and AMG 655 is not restricted to DR5+/DR4− (WM35) cells, and some cells that do not respond to TRAIL or AMG 655 (+protein G) can be sensitized by the combination of TRAIL+AMG 655.

### Example 4—Cooperativity in Lung Tumor Cell Lines that are Sensitive to AMG 655 but Resistant to Trail

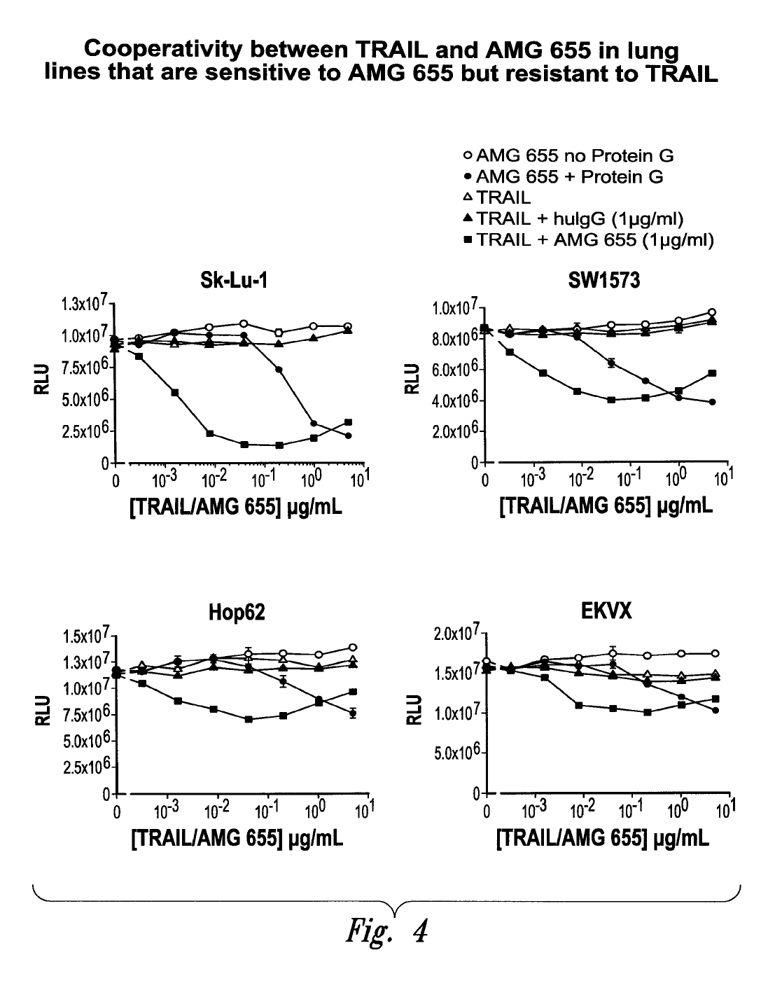

Several cell lines that showed increased sensitivity to AMG 655 (+protein G) vs. TRAIL as single agents were evaluated for their sensitivity to the combination. 2×10\hat{ }4 Sk-Lu-1, SW1573, EKVX, or 1×10\hat{ }4 Hop62 cells/well were plated. The following day, cells were treated with either media, hlgG (1 μg/mL) or AMG 655(1 μg/mL). Cells were then treated with a dose titration of TRAIL or AMG 655 with protein G (1 μg/mL) as indicated (FIG. 4). After a further 24 hour incubation, cell viability was determined using the CellGlo® assay system. Data shown is expressed as relative luminescence units. This experiment was repeated at least 3 times with similar results. The results in FIG. 4 show that some cells that do not respond to TRAIL can be sensitized by the combination of TRAIL+AMG 655. One of the cell lines that showed the most polarized response was SK-Lu-1. It was confirmed that SK-Lu-1 do not respond to soluble TRAIL but are responsive to AMG 655 (+protein G). Despite being unresponsive to soluble TRAIL alone, TRAIL+AMG 655 induced a strong apoptotic response that was more potent than that seen with AMG 655 (+protein G) alone. A total of 8 cell lines were tested from this response group (AMG 655>TRAIL) and all show a strong cooperative interaction between AMG 655 and TRAIL.

### Example 5—Cooperativity Between Trail and AMG 655 in H838 Lung Cancer Cells Leads to Increased Total Cell Death

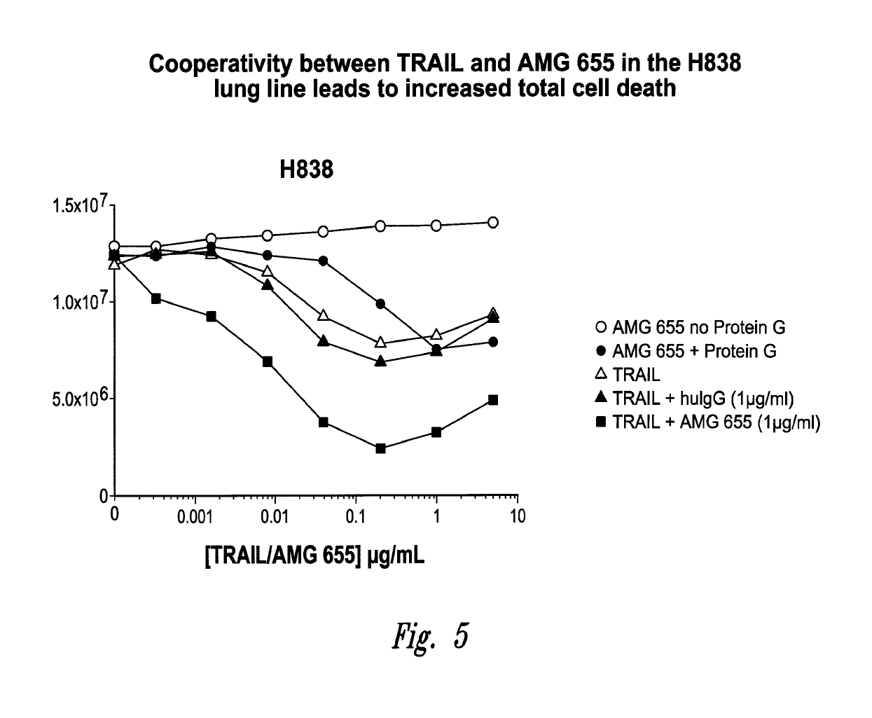

In the majority of cell lines tested, cooperativity between TRAIL and AMG 655 shows increased potency (greater cell killing with lower amounts of drug) but does not necessarily result in greater overall cell killing. H838 lung cancer cells are one example where the combination of TRAIL and AMG 655 have increased potency as well as increased overall cell killing. In this experiment, 1×10\hat{ }4 H838 cells/well were plated. The following day, cells were treated with either media, hIgG (1 μg/mL) or AMG 655(1 μg/mL). Cells were then treated with a dose titration of TRAIL or AMG 655 with protein G (1 μg/mL) as indicated (FIG. 5). After a further 24 hour incubation, cell viability was determined using the CellGlo® assay system. Data shown is expressed as relative luminescence units. This experiment was repeated at least 3 times with similar results. The results in FIG. 5 show that in H383 cells, cooperativity with TRAIL and AMG655 leads to increased potency as well as increased total cell killing.

### Example 6—No Enhanced Cooperativity of Trail+AMG 655 on Normal Cells

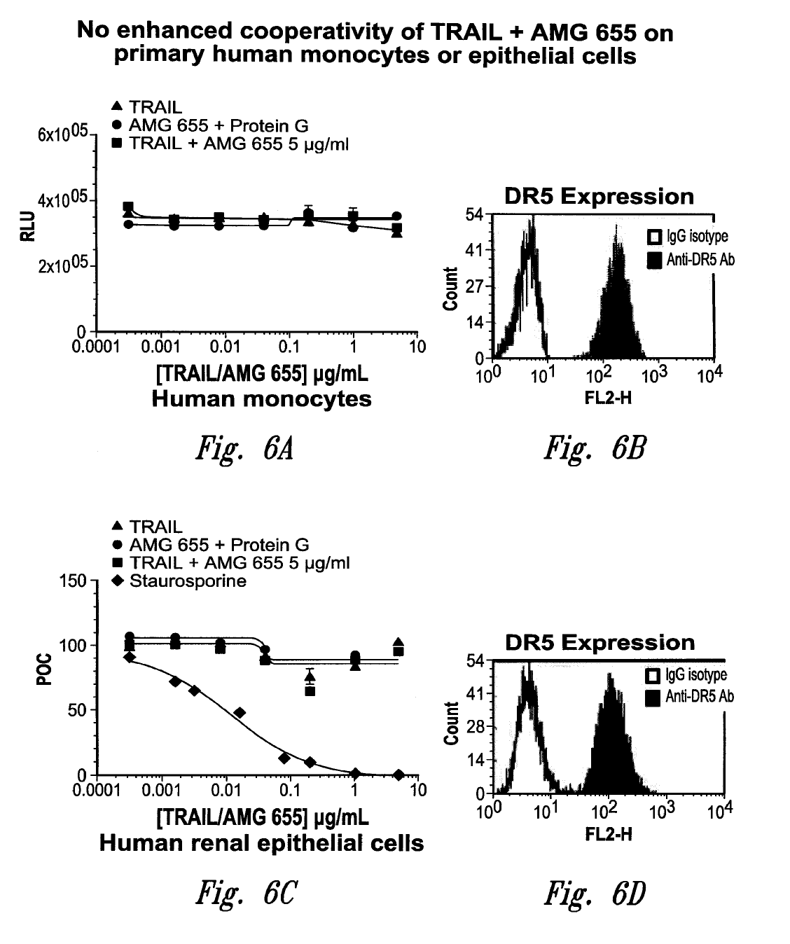

Having shown that TRAIL+AMG 655 is a more potent agonist than either single agent against multiple tumor cell lines, whether this enhanced sensitivity extended to primary normal cells was determined. For one experiment, 1×10\hat{ }5 primary human monocytes, prepared by negative selection from human PBMC (peripheral blood mononuclear cell) leukopacks, were plated at 1×10\hat{ }6/mL 24 hours before being treated with a dose titration of TRAIL or AMG 655+protein G (1 μg/mL). The indicated cells then received AMG 655 (5 μg/mL) (FIG. 6). Cells were analyzed for viability 48 hr after treatment using the ATPLite® luminescence ATP detection assay system (PerkinElmer, USA). Data shown is expressed as relative luminescence units. DR5 expression was confirmed by flow cytometry as described in Example 2. The results in FIGS. 6A and 6B show that primary human monocytes do not respond to either TRAIL or AMG 655 (+protein G) alone, or the combination, despite expressing surface levels of DR5. Similar results were observed in cells obtained from 4 independent donors.

In a related experiment, commercially available primary human renal epithelial cells (Lonza, Walkersville, Md.) were plated at 2×10\hat{ }4 cells/well 24 hours before being treated with a dose titration of TRAIL, AMG 655+protein G (1 μg/mL), or staurosporine. The cells then received AMG 655 (5 μg/mL). After a 24 hour incubation, cell viability was determined by the ATPlite® luminescence ATP detection assay system (PerkinElmer, USA). Data was expressed as percent of control relative to untreated cells. DR5 expression was confirmed by flow cytometry as described in Example 2. As shown in FIG. 6C, human primary renal epithelial cells were insensitive to either TRAIL, AMG 655 (+protein G) or the combination, although they were sensitive to increased amounts of staurosporine. The lack of sensitivity was not due to poor expression of DR5, as shown in FIG. 6D. This experiment was repeated twice with similar results.

Since previous reports have demonstrated that different versions of recombinant TRAIL elicit differential hepatocyte toxicity (Lawrence et al, 2001) the sensitivity of TRAIL, AMG 655 or the combination in primary human hepatocytes from three different donors was evaluated. As expected, both TRAIL and AMG 655 (+protein G) showed some dose-dependent killing activity against hepatocytes (data not shown). Confirming previous observations, LZ-TRAIL was more potent than either TRAIL or AMG 655 (+protein G). Although the combination of TRAIL+AMG 655 was more potent than TRAIL alone, maximal killing of the combination was consistently less than AMG 655+(protein G) or LZ-TRAIL. This suggests that in vitro, hepatocytes have an intermediate response to the combination of TRAIL and AMG 655 (+protein G) as compared to either agent alone.

### Example 7—A Subset of Anti-Dr5 Antibodies Cooperate with Trail

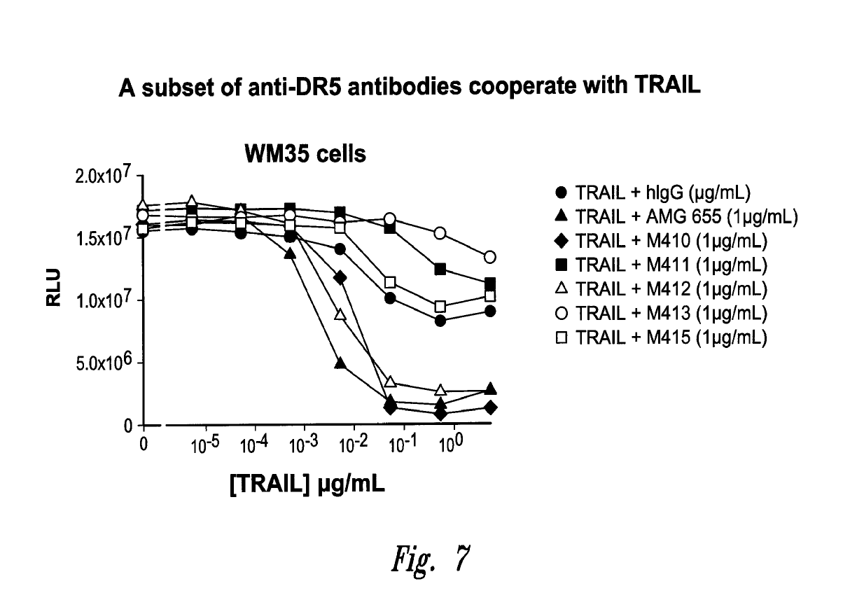

Whether other anti-DR5 antibodies in addition to AMG 655 showed a cooperative relationship with TRAIL was tested. Additional antibodies tested were the mouse anti-human DR5 monoclonal antibodies M410, M411, M412, M413, and M415. In this experiment, 2×10\hat{ }4 WM35 cells/well were plated. The following day, cells were treated with either hlgG (1 μg/mL) or the indicated DR5 antibody (1 μg/mL) (FIG. 7). Cells were then treated with a dose titration of TRAIL. After a further 24 hour incubation, cell viability was determined using the CellGlo® assay system. Data is expressed as relative luminescence units This experiment was repeated at least three times with similar results. FIG. 7 shows that treatment of WM35 cells with M411 or M413, which had previously been shown to compete with TRAIL for binding to DR5 (data not shown), blocked TRAIL-mediated killing. M415 neither competed nor cooperated with TRAIL. However, M410 and M412 showed cooperative behavior with TRAIL that was similar to that observed with AMG 655. These results show that not all anti-DR5 antibodies are capable of cooperating with TRAIL. The anti-DR5 mAb M413 antagonized TRAIL-mediated killing, suggesting that antibodies that compete with ligand for binding to DR5 are unlikely to demonstrate synergy with TRAIL.

### Example 8—Cooperativity of Anti-DR5 Antibody Pairs

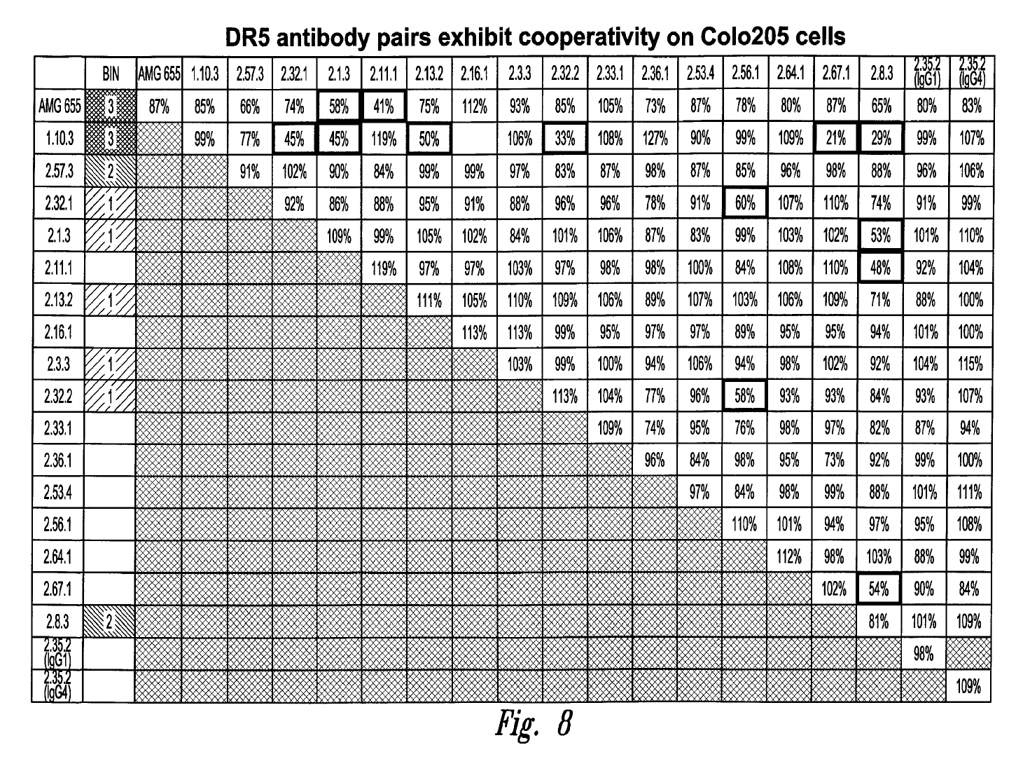

Whether antibodies from the pool from which AMG 655 was originally picked showed evidence of cooperativity with AMG 655 or with one another was addressed. A series of 17 available anti-DR5 IgG2 (unless otherwise indicated) antibodies and AMG 655 were tested for their ability to cooperate with one another in cell viability assays. In these experiments, 2×10\hat{ }4 Colo205 cells/well were plated and 5 hours later, cells were treated with 1 μg/mL each of the indicated DR5 antibodies (FIG. 8). After a further 24 hour incubation, cell viability was determined using the CellGlo® assay system. Data in the table is expressed as percent remaining viable cells. Black boxes indicate those antibody pairs that demonstrated the highest degree of cooperativity. Bin numbers correspond to unique epitope bins. Binning information was not available for all antibodies. The results shown in FIG. 8 indicate that only a subset of antibodies were capable of showing cooperativity with each other, similar to the findings in FIG. 7. Antibody 1.10.3 was capable of cooperating with multiple other antibodies, as indicated by the black boxes showing remaining viable cells. The antibody pair 1.10.3 and 2.67.1 showed the greatest cooperativity, resulting in 21% viable cells remaining at the end of the experiment. Although antibodies 2.1.3 and 2.11.1 showed some cooperativity with AMG 655, neither of these combinations was as potent as the 1.10.3 and 2.67.1 pair.

### Example 9—Cooperativity of Anti-DR5 Antibody Pairs and Anti-DR5 Antibodies with Trail

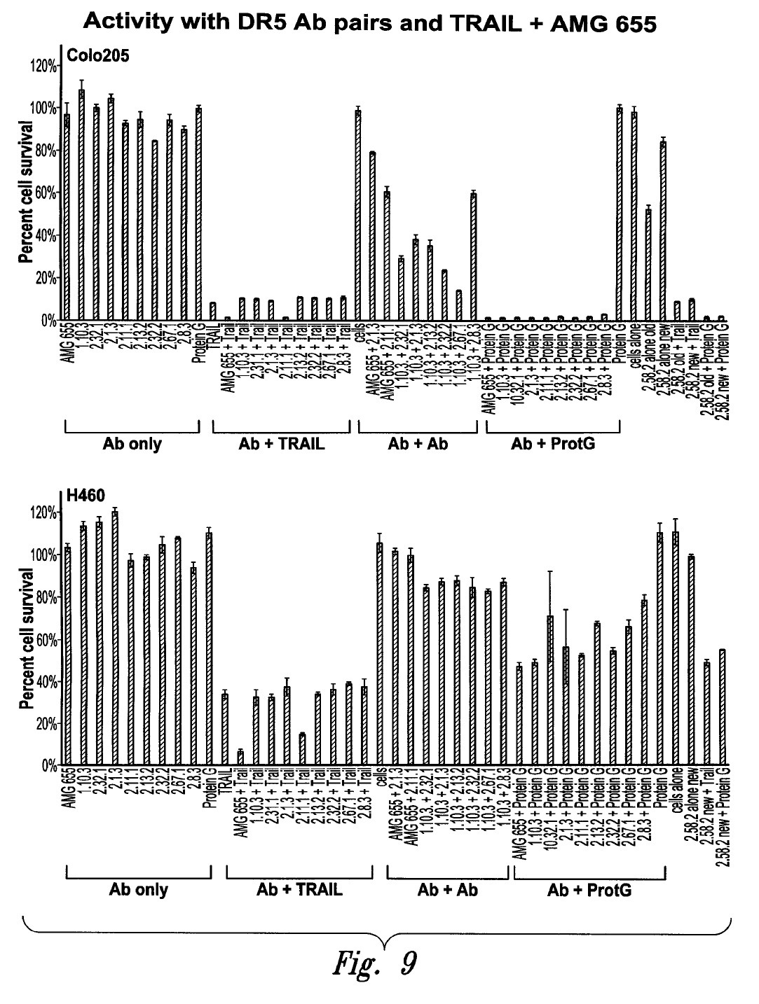

The series of anti-DR5 antibodies from the original AMG 655 pool was tested for their ability to cooperate with TRAIL, and on an additional cell line (H460). In these experiments, 2×10\hat{ }4 Colo205 or H460 cells/well were plated and 5 hours later, cells were treated with either 5 μg/mL each of the indicated DR5 antibodies alone (Ab only); 5 μg/mL of each antibody with 1 μg/mL TRAIL (Ab+TRAIL); 5 μg/mL each indicated antibody pair (Ab+Ab); or antibody+1 μg/mL Protein G (FIG. 9). After a further 24 hour incubation, cell viability was determined using the CellGlo® assay system. Data is expressed as percent cell survival. The results in FIG. 9 show that in both cell lines all of the antibodies show little or no killing activity in the absence of Protein G cross-linker, but all show some activity in the presence of Protein G. In both cell lines, both AMG 655 and 2.11.1 show cooperativity with TRAIL. The 1.10.3 and 2.67.1 antibody pair shows the greatest cooperativity on Colo205 cells, but only displays limited activity on H460 cells. Although the 1.10.3 and 2.67.1 antibody pair show good cooperativity with one another, neither antibody was capable of cooperating with TRAIL, suggesting that these antibodies may share an overlapping epitope with TRAIL.

### Example 10—Cooperativity of the 1.10.3 and 2.67.1 Anti-DR5 Antibody Pair in a COLO205 Xenograft Model

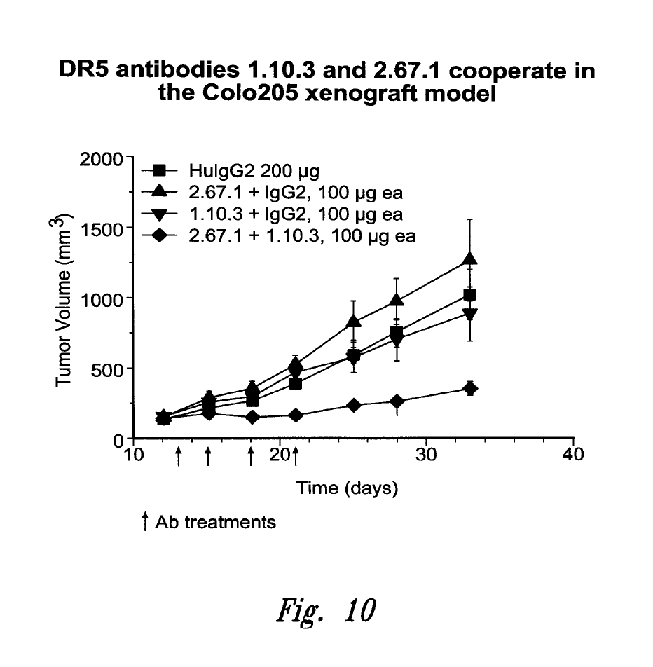

Whether the antibody pair that showed the greatest cooperativity in vitro (see FIG. 8) could also show enhanced anti-tumor activity in a tumor xenograft model was determined. Female NOD-SCID (Non-Obese Diabetic-Severe Combined ImmunoDeficiency) mice approximately 8 weeks of age were injected subcutaneously (s.c.) in the right flank with 1×10⁶ Colo205 cells. Thirteen days following tumor cell implantation, mice were treated with either human IgG2 (200 μg, n=10/group), 2.67.1 (IgG2 isotype)+IgG2 (100 μg each, n=8/group), 1.10.1(IgG2 isotype)+IgG2 (100 μg each, n=8/group), or 1.10.1+2.67.1 (100 μg each, n=8/group) (FIG. 10). All antibodies were administered via intraperitoneal injection (i.p.) twice per week for 2 weeks. Tumor dimensions were measured 2×/week by a digital caliper and tumor volumes were calculated as length×(width)²/2. As shown in FIG. 10, no anti-tumor efficacy was observed with either 1.10.1 or 2.67.1 antibody alone. However, significant tumor growth inhibition was observed when 1.101 and 2.67.1 were combined (p<000.1). These results indicate that 1.10.1 and 2.67.1 can cooperate in vivo and that this cooperativity is independent of FcγR-mediated cross-linking, as IgG2 antibodies have low or no affinity for Fc receptor subtypes.

1. A composition for inducing DR5-mediated apoptosis of a cancer cell in a mammal, said composition comprising:
a) a first anti-DR5 binding polypeptide, wherein said first anti-DR5 binding polypeptide specifically binds to the extracellular domain of a DR5 receptor of said cancer cell; and,
b) at least one of:
i. a second anti-DR5 binding polypeptide, wherein said second anti-DR5 binding polypeptide specifically binds to said extracellular domain of said DR5 receptor, and wherein said first binding polypeptide and said second binding polypeptide do not competitively inhibit each other from specifically binding to said DR5 receptor, and wherein specific binding of said first anti-DR5 binding polypeptide and said second anti-DR5 binding polypeptide cooperatively induce apoptosis of said mammalian cancer cell without requiring natural killer (NK) cells of said mammal; or,
ii. a tumor necrosis factor related apoptosis inducing ligand (TRAIL), wherein said TRAIL specifically binds to said DR5 receptor, and wherein said first binding polypeptide and said TRAIL do not competitively inhibit each other from specifically binding to said DR5 receptor, and wherein specific binding of said first anti-DR5 binding polypeptide and said TRAIL cooperatively induce apoptosis of said mammalian cancer cell without requiring natural killer (NK) cells of said mammal.

2. The composition of claim 1, wherein said mammalian cancer cell is a human cancer cell.

3. The composition of claim 1, wherein said first anti-DR5 binding polypeptide or said second anti-DR5 binding polypeptide is an antibody.

4. The composition of claim 1, wherein said first anti-DR5 binding polypeptide and said second anti-DR5 binding polypeptide are each antibodies.

5. The composition of claim 4, wherein said antibodies are each a fully human antibody.

6. (canceled)

7. The composition of claim 5, wherein said first anti-DR5 antibody or said second anti-DR5 antibody induces apoptosis in Colo205 cells in vitro without cross-linking.

8. The composition of claim 5, wherein said mammalian DR5 receptor is a human DR5 receptor.

9. (canceled)

10. The composition of claim 1, wherein at least one of said first anti-DR5 binding polypeptide or said second anti-DR5 binding polypeptide do not specifically bind to FCGR3A of said NK cells.

11. The composition of claim 1, wherein said first anti-DR5 binding polypeptide in combination with said second anti-DR5 binding polypeptide or said TRAIL cooperatively induce apoptosis.

12. The composition of claim 1, comprising said first anti-DR5 binding polypeptide and said TRAIL.

13. The composition of claim 11, wherein TRAIL is LZ-TRAIL.

14. The composition of claim 11 formulated in a pharmaceutically acceptable carrier.

15. The composition of claim 11, wherein said first anti-DR5 binding polypeptide is an antibody.

16. (canceled)

17. The composition of claim 14, wherein said antibody is a human IgG1 antibody comprising a N297A substitution.

18. The composition of claim 14, wherein at least one of said first anti-DR5 binding polypeptide or said second anti-DR5 binding polypeptide comprises an aglycosylated Fc.

19. The composition of claim 14, wherein said first anti-DR5 binding polypeptide is an antibody and said antibody is AMG 655.

20. (canceled)

21. (canceled)

22. A method of inhibiting the growth of DR5 expressing cancer in a mammal, said method comprising administering a therapeutically effective amount of the composition of claim 1.

23. The method of claim 22, wherein said composition is administered in combination with a therapeutically effective amount of a chemotherapeutic agent.

24. The method of claim 22, wherein said cancer is pancreatic cancer, colorectal cancer, or lung cancer.

25. A method of inhibiting the growth of DR5 expressing cancer in a mammal, said method comprising administering a therapeutically effective amount of the composition of claim 14.


SEQUENCE LISTING (8 sequences)

SEQ ID NO: 1 - DNA, 360 bp, Homo sapiens
  CDS (1)..(360)
caggtgcagctgcaggagtcgggcccaggactggtgaagccttcacagaccctgtccctcacctgcactgtctctggtggctccatcagcagtggtggtcactactggagctggatccgccagcacccagggaagggcctggagtggattgggtacatctattacagtgggagcacctactacaacccgtccctcaagagtcgagttaccatatcagtagacacgtctaagaaccagttctccctgaagctgagctctgtgactgccgcggacacggccgtgtattattgtgcgagagatgacagcagtggctggggttttgactactggggccagggaatcctggtcaccgtctcctca

SEQ ID NO: 2 - protein, 120 aa, Homo sapiens
QVQLQESGPGLVKPSQTLSLTCTVSGGSISSGGHYWSWIRQHPGKGLEWIGYIYYSGSTYYNPSLKSRVTISVDTSKNQFSLKLSSVTAADTAVYYCARDDSSGWGFDYWGQGILVTVSS

SEQ ID NO: 3 - DNA, 324 bp, Homo sapiens
  CDS (1)..(324)
gacatccagatgacccagtctccatcctccctgtctgcatctgttggagacagagtcaccatcacttgccgggcaagtcagggccttagaaatgatttaggctggtttcagcagaaaccagggaaagtcactaagcgcctgatctatgctgcatccagtttgcaaagaggggtcccatcaaggttcagcggcagtggatctgggacagaattcactctcacaatcagcagcctgcagcctgaagattttgcaacttattactgtctacagcattatagtttcccgtggacgttcggccaagggaccaaggtggagatcaaacgt

SEQ ID NO: 4 - protein, 108 aa, Homo sapiens
DIQMTQSPSSLSASVGDRVTITCRASQGLRNDLGWFQQKPGKVTKRLIYAASSLQRGVPSRFSGSGSGTEFTLTISSLQPEDFATYYCLQHYSFPWTFGQGTKVEIKR

SEQ ID NO: 5 - DNA, 363 bp, Homo sapiens
  CDS (1)..(363)
gaggtgcagctggtggagtctgggggaggcctggtcaagcctggggggtccctgagactctcctgtgcagcctctggattcaccttcagtagctatagcatgaactgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagagccgaggacacggctgtgtattactgtgcgggggggagcagtggctggtacggggggtggttcgacccctggggccagggaaccctggtcaccgtctcctca

SEQ ID NO: 6 - protein, 121 aa, Homo sapiens
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCAGGSSGWYGGWFDPWGQGTLVTVSS

SEQ ID NO: 7 - DNA, 324 bp, Homo sapiens
  CDS (1)..(324)
gacatccagatgacccagtctccatcctccctgtctgcatctgtaggagacagagtcaccatcacttgccgggcaagtcagagcattagcggctatttaaattggtatcagcagaaaccagggaaagcccctaaactcctgatctacgctgcatccagtttgcaaagtggggtcccatcaaggttcagtgacagtggatctgggacagatttcactctcaccatcagcagtctgcaacctgaagattttgcaacttactactgtcaacagagttacagaaccccattcactttcggccctgggaccaaagtggatatcaaacgt

SEQ ID NO: 8 - protein, 108 aa, Homo sapiens
DIQMTQSPSSLSASVGDRVTITCRASQSISGYLNWYQQKPGKAPKLLIYAASSLQSGVPSRFSDSGSGTDFTLTISSLQPEDFATYYCQQSYRTPFTFGPGTKVDIKR